(19) United States
(12) Patent Application Publication
(10) Pub. No.: US 2008/0032964 A1
(43) Pub. Date: Feb. 7, 2008

(54) Process for the synthesis of azetidinone

(76) Inventors: Vinod Kumar Kansal, Haryana (IN); Suhail Ahmad, New Delhi (IN); Shanmugavel Mariappan, Tamilnadu (IN); Bhupendra Tyagi, Uttaranchal (IN); Nurit Perlman, Kfar Saba (IL); Jacques Le Paih, Cambridge (GB); Antonio Zanotti-Gerosa, Cambridge (GB)

Correspondence Address: KENYON & KENYON LLP, ONE BROADWAY, NEW YORK, NY, 10004, US

(21) Appl. No.: 11/786,449

(22) Filed: Apr. 10, 2007

Related U.S. Application Data
(60) Provisional application No. 60/791,114, filed on Apr. 10, 2006.
(60) Provisional application No. 60/831,908, filed on Jul. 18, 2006.
(60) Provisional application No. 60/897,689, filed on Jan. 25, 2007.

(51) Int. Cl. A61K 31/397; A61P 3/06; C07C 31/20; C07D 205/04; C07D 317/26; C07D 319/06; C07D 321/06; C07D 321/12; C07D 405/06; C07D 413/06; C07D 417/06

(52) U.S. Cl. 514/210.02; 540/200; 548/188; 548/230; 548/311.1; 549/347; 549/375; 549/454; 568/852

(57) ABSTRACT

Provided are intermediates useful for the synthesis of hydroxyl-alkyl substituted azetidinones, processes of their preparation, and processes for the synthesis of certain hydroxyl-alkyl substituted azetidinones. Also provided are processes for the synthesis of 1-(4-fluorophenyl)-3(R)-[3-(4-fluorophenyl)-3(S)-hydroxypropyl]-4(S)-(4-hydroxyphenyl)-2-azetidinone, or ezetimibe.

### CROSS REFERENCE TO RELATED APPLICATIONS

This application claims the benefit of U.S. Ser. No. 60/791,114, filed Apr. 10, 2006, Ser. No. 60/831,908, filed Jul. 18, 2006, and Ser. No. 60/897,689, filed Jan. 25, 2007, the contents of all of which are incorporated herein by reference in their entirety.

### FIELD OF THE INVENTION

The invention relates to a process for the synthesis of certain hydroxyl-alkyl substituted azetidinones. More particularly, the invention relates to a novel process for the synthesis of 1-(4-fluorophenyl)-3(R)-[3-(4-fluorophenyl)-3(S)-hydroxypropyl]-4(S)-(4-hydroxyphenyl)-2-azetidinone i.e. ezetimibe.

### BACKGROUND OF THE INVENTION

Hydroxyl-alkyl substituted azetidinones useful as hypocholesterolemic agents in the treatment and prevention of atherosclerosis, in particular, 1-(4-fluorophenyl)-3(R)-[3-(4-fluorophenyl)-3(S)-hydroxypropyl]-4(S)-(4-hydroxyphenyl)-2-azetidinone, i.e. ezetimibe. Ezetimibe is a selective inhibitor of intestinal cholesterol and related phytosterol absorption. The empirical formula for ezetimibe is C₂₄H₂₁F₂NO₃, and its molecular weight is 409.4 g/mol. Ezetimibe is a white, crystalline powder that is freely to very soluble in ethanol, methanol and acetone and practically insoluble in water. Ezetimibe has the following chemical structure:

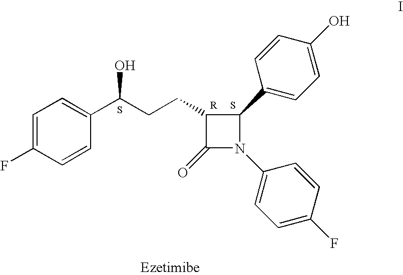

Ezetimibe is the active ingredient sold under the name ZETIA®, manufactured by Merck/Schering-Plough Pharmaceuticals, and is approved by the United States Food and Drug Administration for use in patients with high cholesterol to reduce LDL cholesterol and total cholesterol.

Different processes for preparing 1-(4-fluorophenyl)-3(R)-[3-(4-fluorophenyl)-3(S)-hydroxypropyl]-4(S)-(4-hydroxyphenyl)-2-azetidinone i.e. ezetimibe are disclosed in U.S. Pat. Nos. 5,631,365; 5,739,321; 5,856,473; 5,767,115 and 6,207,822. However, there are several drawbacks associated with the processes describe in the art. These drawbacks include the use of pyrophoric bases, such as n-butyl lithium and a metalamide, e.g., LDA, and low temperatures, e.g., below −50° C., which lead to difficulties in preparation of ezetimibe on a commercial scale. Other drawbacks include the use of Grignard reactions and zincate, which are sensitive to moisture and difficult to prepare, and the use of column chromatography for purification, which may not be feasible on a commercial scale. In addition, most of the intermediate compounds produced in these reactions are in liquid form, which makes purification process more difficult.

Thus, there is a pressing need in the art for new processes for the preparation of azetidinone, particularly ezetimibe that does not suffer from the above mentioned disadvantages.

### SUMMARY OF INVENTION

In one embodiment, the present invention encompasses a compound of Formula XII: 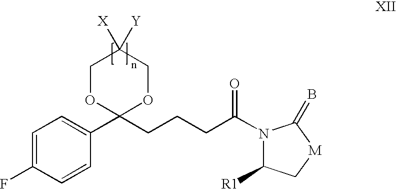
wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl, with the proviso that X and Y are not both hydrogen.

In another embodiment, the invention encompasses a process for preparing the compound of Formula XII comprising (a) combining the compound of Formula XIII: 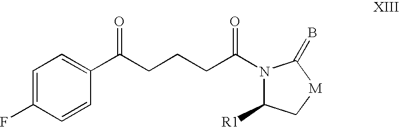
with a diol derivative of Formula III: 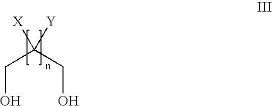
to obtain the compound of Formula XII, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl. Preferably, X and Y for the compound of Formula XII are not both hydrogen.

The present invention encompasses a process for preparing the compound of Formula XII comprising combining a compound of Formula VIII: 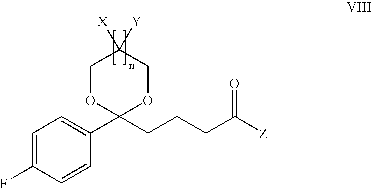
with a chiral auxiliary of Formula IX: 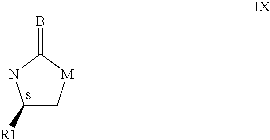
to form the compound of Formula XII, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl; Z is a halogen, an activated ester of a carboxylic acid, —O—CO—R or —O—COOR; and R is C₁₋₈ alkyl. Preferably, X and Y for the compound of Formula XII are not both hydrogen.

In another embodiment, the invention encompasses a process for preparing the compound of Formula XII comprising (a) reacting a 4-fluorobenzoyl butyric acid or ester of Formula V: 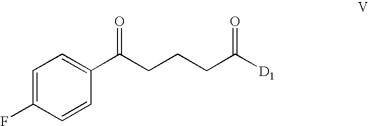
with a diol derivative of Formula III: 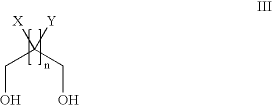
to form a compound of Formula VI: 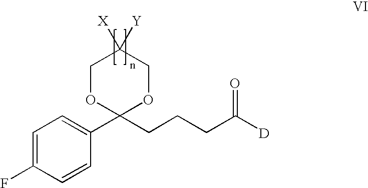
(b) reacting the compound of Formula VI with an inorganic base to obtain a compound of Formula VII: 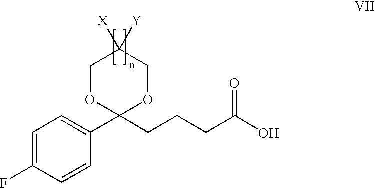
(c) converting the compound of Formula VII to a compound of Formula VIII: 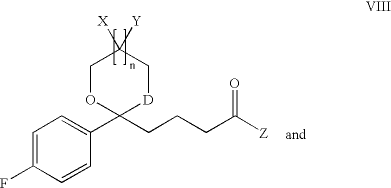
(d) reacting the compound of Formula VIII with a chiral auxiliary of Formula IX: 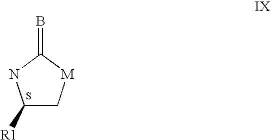
to obtain a compound of Formula XII, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl; Z is a halogen, an activated ester of a carboxylic acid, —O—CO—R or —O—COOR; and R is C₁₋₈ alkyl; D is substituted or unsubstituted C₁₋₈ alkoxy, and D₁ is hydroxyl, or substituted or unsubstituted C₁₋₈ alkoxy. Preferably, X and Y for the compound of Formula XII are not both hydrogen.

In one embodiment, the invention encompasses a compound of Formula VI: 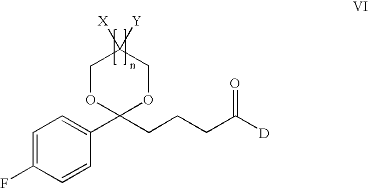
wherein X is hydrogen or a substituted or unsubstituted C₁₋₈ alkyl; Y is hydrogen or a substituted or unsubstituted C₁₋₈ alkyl; n is an integer between 0 and 3; and D is substituted or unsubstituted C₁₋₈ alkoxy.

In another embodiment, the invention encompasses a process for preparing the compound of Formula VI comprising reacting a 4-fluorobenzoyl butyric acid or ester of Formula V: 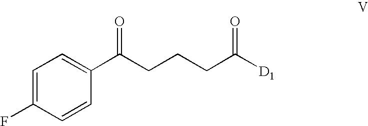
wherein D₁ is hydroxyl, substituted or unsubstituted C₁₋₈ alkoxy, with a diol derivative of Formula III: 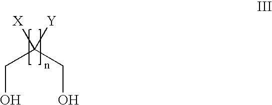
to obtain a compound of Formula VI.

The present invention also encompasses a compound of Formula VII: 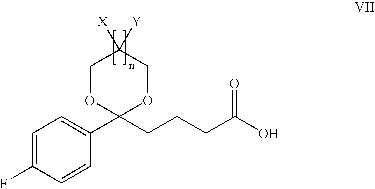
wherein X is hydrogen or a substituted or unsubstituted C₁₋₈ alkyl; Y is hydrogen or a substituted or unsubstituted C₁₋₈ alkyl; and n is an integer between 0 and 3.

In another embodiment, the invention encompasses a process for preparing the compound of Formula VII comprising reacting a compound of Formula VI: 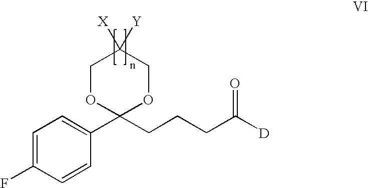
with an inorganic base to obtain the compound of Formula VII, wherein
D is substituted or unsubstituted C₁₋₈ alkoxy.

The present invention also encompasses a compound of Formula VIII: 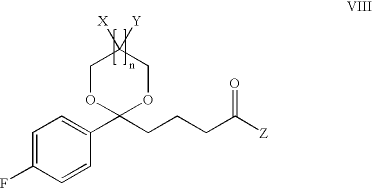
wherein X is hydrogen or a substituted or unsubstituted C₁₋₈ alkyl; Y is hydrogen or a substituted or unsubstituted C₁₋₈ alkyl; n is an integer between 0 and 3; Z is a halogen, an activated ester of a carboxylic acid, —O—CO—R or —O—COOR; and R is C₁₋₈ alkyl.

In another embodiment, the present invention encompasses a process for preparing the compound of Formula VIII comprising reacting a compound of Formula VII: 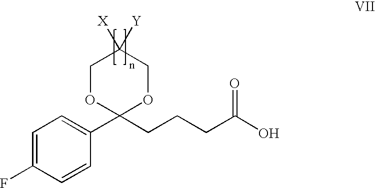
with a reagent selected from the group consisting of thionyl chloride, thionyl bromide, oxalyl chloride, oxalyl bromide, phosphorous penta chloride, phosphorus penta bromide, phosphorous oxychloride, chloroformates, pivolyl chloride, pivolyl bromide, dicyclohexylcarbodiimide, N-hydroxy succinimde, and 1-hydroxybenzotriazole to form the compound of Formula VIII.

In another embodiment, the present invention encompasses a compound of Formula X: 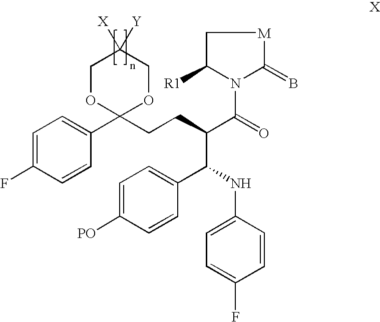
wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; and P is a hydroxyl protecting group, with the proviso that X and Y are not both hydrogen.

In another embodiment, the present invention encompasses a process for preparing the compound of Formula X comprising reacting a compound of Formula XII: 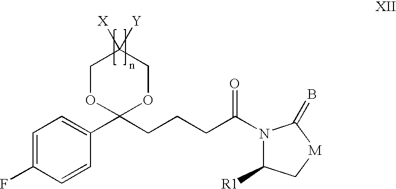
with a compound of Formula XI: 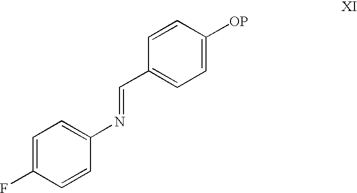
in the presence of a tertiary organic base and at least one acid catalyst selected from the group consisting of para-toluene sulfonic acid, methane sulfonic acid, benzene sulfonic acid, C₁₋₄ tri-alkylsilyl chloride, e.g., trimethyl silyl chloride, titanium tetrachloride, titanium tetra isopropoxide, aluminum chloride, zinc chloride, and BF₃-etherate to obtain the compound of Formula X, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; and P is a hydroxyl protecting group. Preferably, X and Y for the compound of Formula XII and the compound of Formula X are not both hydrogen.

In one embodiment, the present invention encompasses a compound of Formula IV: 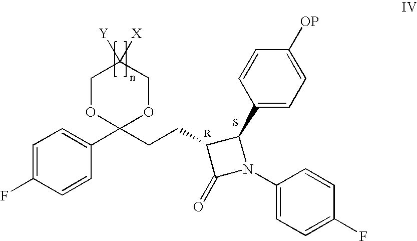
wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group, with the proviso that X and Y are not both hydrogen.

In another embodiment, the invention encompasses a process for preparing the compound of Formula IV comprising cyclizing a compound of Formula X: 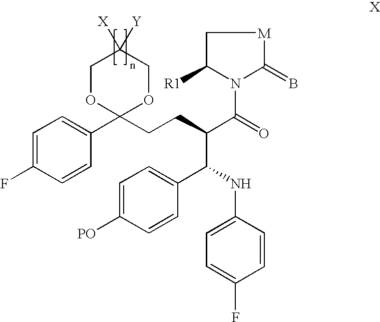
with a silylating agent selected from the group consisting of bis(trimethylsilyl)acetamide, N-methyl-O-trimethylsilyl acetamide, iso-propenyloxy trimethylsilane, bis(trimethylsilyl)urea, hexamethyldisilazane, and a mixture of hexamethyldisilazane and trimethyl silyl chloride, optionally in the presence of a fluoride ion catalyst, to form the compound of Formula IV, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group, B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl. Preferably, X and Y for the compound of Formula X and the compound of Formula IV are not both hydrogen.

In one embodiment, the invention encompasses a compound of Formula XV: 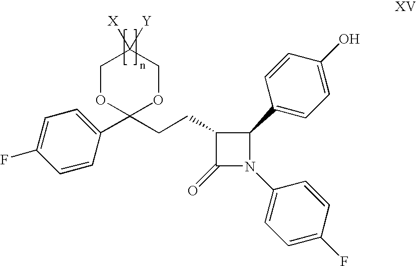
wherein X is hydrogen or a substituted or unsubstituted C₁₋₈ alkyl; Y is hydrogen or a substituted or unsubstituted C₁₋₈ alkyl; and n is an integer between 0 and 3, with the proviso that X and Y are not both hydrogen.

In another embodiment, the invention encompasses a process for preparing the compound of Formula XV comprising converting a compound of Formula IV: 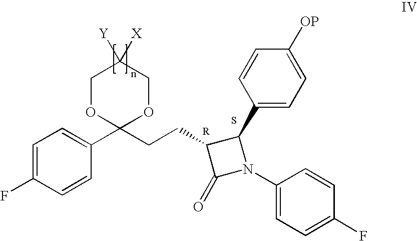
to the compound of Formula XV by removing P using catalytic hydrogenation or hydride transfer reaction, wherein P is a hydroxyl protecting group; X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula XV and the compound of Formula IV are not both hydrogen.

In another embodiment, the invention encompasses a process for preparing the compound of Formula XV comprising reacting a compound of Formula IV: 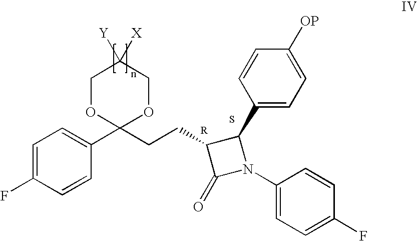
with a quaternary ammonium fluoride in the presence of an organic solvent to obtain the compound of Formula XV, wherein P is a hydroxyl protecting group, X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula XV and the compound of Formula IV are not both hydrogen.

In one embodiment, the invention encompasses a process for preparing a compound of Formula XVI: 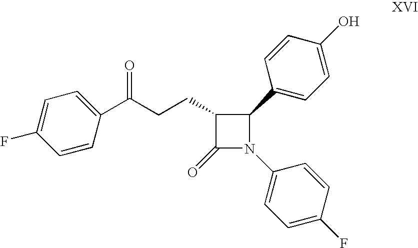
comprising converting a compound of Formula IV: 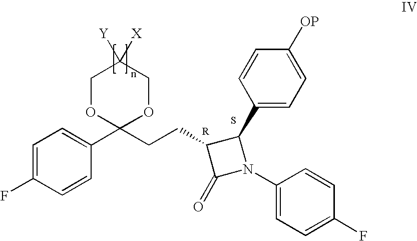
to the compound of Formula XVI using acid hydrolysis, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group. Preferably, X and Y for the compound of Formula IV are not both hydrogen.

In another embodiment, the invention encompasses a process for preparing a compound of Formula XVI comprising converting the compound of Formula XV: 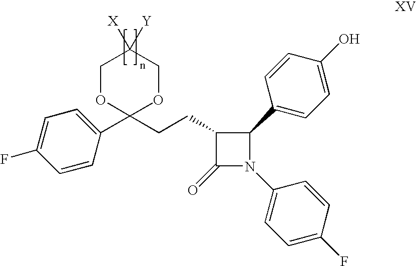
to the compound of Formula XVI in the presence of at least one of an organic acid or a mineral acid, wherein X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula XV are not both hydrogen.

In another embodiment, the invention encompasses a process for preparing hydroxyl-alkyl substituted azetidinones, particularly ezetimibe of Formula I: 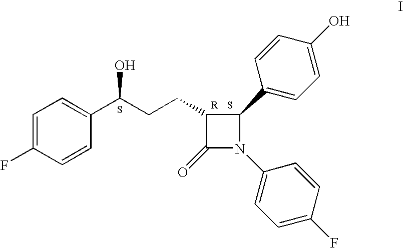
comprising converting a compound of Formula XII, VI, VII, VIII, X, IV, or XV to the azetidinone. Preferably, X and Y for the compounds of Formula XII, IV, X, and XV are not both hydrogen.

In another embodiment, the invention encompasses a process for preparing hydroxyl-alkyl substituted azetidinones, particularly ezetimibe, comprising:
(a) deprotecting a compound of Formula IV: 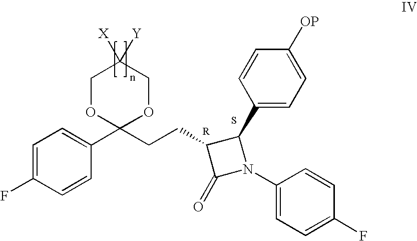
using catalytic hydrogenation, hydride transfer reduction, acid hydrolysis, or reacting with a quaternary ammonium fluoride to obtain a compound of Formula XV: 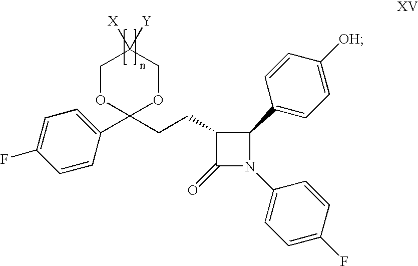
(b) deprotecting the compound of Formula XV with an acid to obtain a compound of Formula XVI: 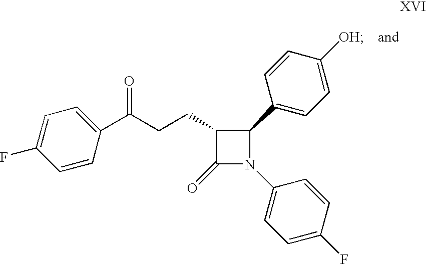
(c) reducing the compound of Formula XVI in the presence of a chiral catalyst to ezetimibe, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group. Preferably, X and Y for the compounds of Formula IV, and XV are not both hydrogen.

In another embodiment, the invention encompasses a process for preparing hydroxyl-alkyl substituted azetidinones, particularly ezetimibe, comprising at least one of the following steps:
(a) reacting a compound of Formula XIII: 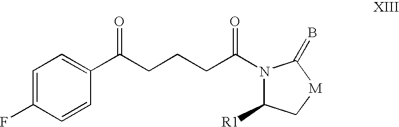
with a diol derivative of Formula III: 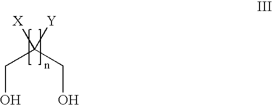
to obtain a compound of Formula XII: 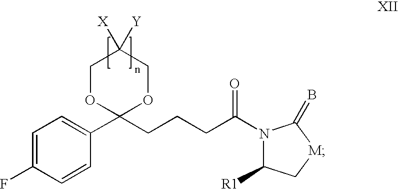
(b) reacting the compound of Formula XII with a compound of Formula XI: 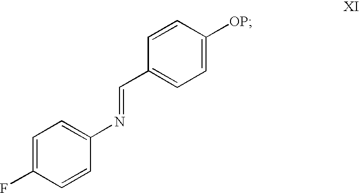
to obtain a compound of Formula X: 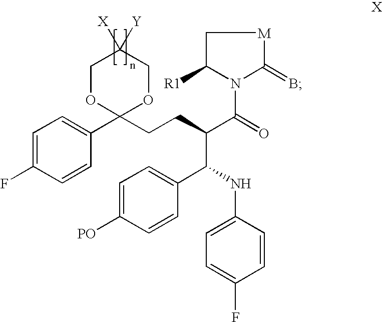
(c) combining the compound of Formula X with a silylating agent and optionally with a fluoride ion catalyst of Formula XVII: 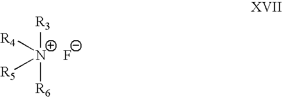
to obtain a compound of Formula IV: 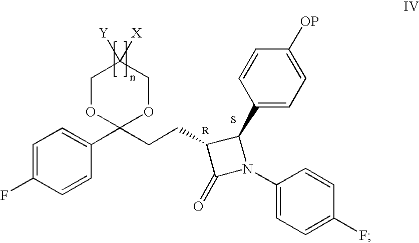
(d) combining the compound of Formula IV with an acid to obtain a compound of Formula II: 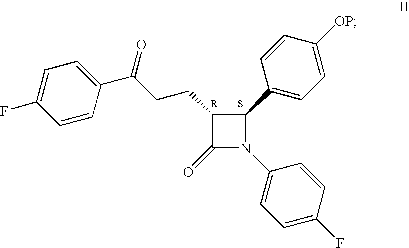
(e) converting the compound of Formula II to the compound of Formula XIV:

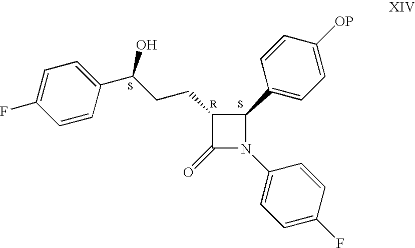

by (i) combining the compound of Formula II with at least one chiral reducing agent selected from the group consisting of β-chloro diisopinocamphenyl borane, borane, borane-methyl sulfide complex, borane-morpholine complex, borane-pyridine complex, borane-tetrahydrofuran complex, borane-tributylphosphine complex, borane-triethylamine complex, borane-trimethylamine complex, borane-1,4 thioxane, borane-dimethylsulfide complex, borane 1,4-dioxane, borane diethylaniline, borane N-ethyl-N-isopropylaniline, N-borane phenylamine, and catecholborane in the presence of at least one chiral catalyst selected from the group consisting of:
(R)-tetrahydro-1-methyl-3,3-diphenyl-1H,3H-pyrrol(1,2-c)(1,3,2)oxaza-borolideine,
(R)-tetrahydro-1-butyl-3,3-diphenyl-1H,3H-pyrrol(1,2-c)(1,3,2)oxaza-borolideine, and
(R)-tetrahydro-1-phenyl-3,3-diphenyl-1H,3H-pyrrol(1,2-c)(1,3,2)oxaza-borolideine,
   and optionally at least one solvent selected from the group consisting of:
   dichloromethane, tetrahydrofuran, toluene, 2-methyl tetrahydrofuran, ethyl acetate, and tert-butyl methyl ether, to obtain the compound of Formula XIV;

by (ii) combining the compound of Formula II with at least one chiral catalyst selected from the group consisting of [[RuCl₂(p-cymene)]₂, (S,S)-i-Pr—SO₂DPEN], [[RuCl₂(p-cymene)]₂(S,S)-MsDPEN], [[RuCl₂(hexaMe-benzene)]₂(S,S)-MsDPEN], [[RuCl₂(benzene)]₂(S,S)-MsDPEN], [[RuCl₂(p-cymene)]₂(S,S)-MesithylSO₂-DPEN], [[RuCl₂(p-cymene)]₂(S,S)-2-naphthyl-DPEN], [[RuCl₂(p-cymene)]₂(S,S)-n-Butyl-DPEN], [[RuCl₂(mesitylene)]₂(S,S)-Ms-DPEN], [[RuCl₂(mesitylene)]₂(S,S)-iPr—SO₂-DPEN], [[RuCl₂(hexaMe-benzene)]₂(S,S)-iPr—SO₂-DPEN], [[RuCl₂(p-cymene)]₂(S,S)-n-Bu-SO₂-DPEN], [[RuCl₂(mesitylene)]₂(S,S)-n-Bu-SO₂-DPEN], [[RuCl₂(hexaMe-benzene)]₂(S,S)-n-Bu-SO₂-DPEN], [[RuCl₂(p-cymene)]₂(S,S)-Bn-SO₂-DPEN], [[RuCl₂(p-cymene)]₂(S,S)—Cs-DPEN], [[RuCl₂(p-cymene)]₂(S,S)-3,4-diMe-Ph-SO₂-DPEN], [[RuCl₂(p-cymene)]₂(S,S)-1-Naphthyl-DPEN], [[RuCl₂(p-cymene)]₂(S,S)-1,3,5-tri-iPr-Ph-DPEN], [[RuCl₂(p-cymene)]₂(S,S)-3,4-diMeO-PhSO₂-DPEN], [[RuCl₂(mesitylene)]₂(S,S)—CF₃—SO₂-DPEN], [[RuCl₂(p-cymene)]₂(S,S)-pClPh-SO₂-DPEN], [[RuCl₂(p-cymene)]₂(S,S)-pPh-Ph-SO₂-DPEN], [[RUCl₂(p-cymene)]₂(S,S)-pCF₃-Ph-SO₂-DPEN], [[RuCl₂(p-cymene)]₂(S,S)-pNO₂-Ph-SO₂-DPEN], [[RuCl₂(p-cymene)]₂(S,S)-pMeO-Ph-SO₂-DPEN], [[RuCl₂(p-cymene)]₂(S,S)-Ms-DACH], [[RuCl₂(p-cymene)]₂(S,S)-1-Naphtyl-SO₂-DACH], [(S,S)-Tethered —RuCl], [[RhCl₂Cp*]₂(S,S)-Ms-DPEN], [[RhCl₂Cp*]₂(S,S,S)—Cs-DPEN], and [[RuCl₂(mesitylene)]₂(S,S)-i-Bu-SO₂-DPEN]; at least one hydrogen source selected from the group consisting of formic acid or a salt thereof, C₃-C₁₃ secondary alcohol, and cyclohexadiene; and an organic solvent to obtain the compound of Formula XIV; or

by (iii) combining the compound of Formula II with a chiral catalyst under an inert gas environment; adding a base to obtain a reaction mixture; and subjecting the reaction mixture to a hydrogen pressure of about 2 bars to about 40 bars to obtain the compound of Formula XIV; or

(f) deprotecting group the compound of Formula XIV to obtain the azetidinone, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; P is a hydroxyl protecting group; R₃ is a substituted or unsubstituted C₁₋₈ alkyl or C₆₋₁₄ aryl; R₄ is a substituted or unsubstituted C₁₋₈ alkyl or C₆₋₁₄ aryl; R₅ is a substituted or unsubstituted C₁₋₈ alkyl or C₆₋₁₄ aryl; and R₆ is a substituted or unsubstituted C₁₋₈ alkyl or C₆₋₁₄ aryl. Preferably, X and Y for the compounds of Formula XII, X, and IV are not both hydrogen.

In another embodiment, the invention encompasses a process for preparing a compound of Formula II: 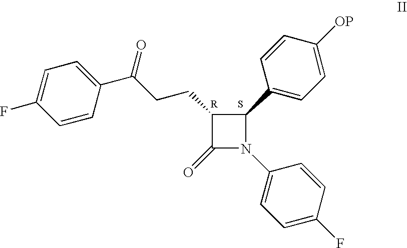
comprising combining a compound of Formula IV: 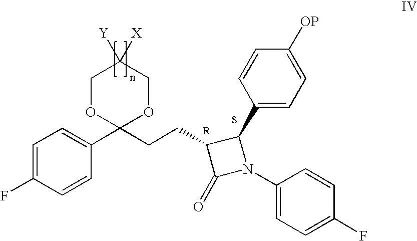
with an acid, preferably at least one of formic acid, acetic acid, propionic acid, camphor sulfonic acid, hydrochloric acid, or sulfuric acid, to obtain the compound of Formula II, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group. Preferably, X and Y for the compound of Formula IV are not both hydrogen.

In another embodiment, the invention encompasses the use of a diol derivative of Formula III: 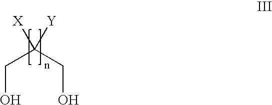
as a carbonyl protecting group for the manufacture of ezetimibe, wherein X is hydrogen or a substituted or unsubstituted C₁₋₈ alkyl; Y is hydrogen or a substituted or unsubstituted C₁₋₈ alkyl; and n is an integer between 0 and 3.

In another embodiment, the present invention encompasses crystalline 3-{4-[2-(4-Fluorophenyl)-5,5-dimethyl-[1,3]dioxan-2-yl]butyryl}-(4S)-phenyl-1,3-oxazolidin-2-one characterized by a PXRD pattern having peaks at about 16.3, 19.5, 20.3, 24.4, and 25.0±0.2 degrees two-theta. Preferably, the crystalline form is further characterized by a PXRD pattern having peaks at about 13.8, 17.5, 26.6, and 28.8 degrees two-theta.

The invention further encompasses compounds prepared according to the processes of the invention, azetidinones prepared therefrom, including ezetimibe, and pharmaceutical compositions comprising such azetidinone. The invention also encompasses a method for reducing cholesterol in a mammal comprising administering a therapeutically effective amount of such azetidinone or such pharmaceutical composition.

### BRIEF DESCRIPTION OF THE FIGURES

FIG. 1 illustrates a PXRD pattern of the crystalline form of the compound of Formula II.

FIG. 2 illustrates PXRD values of the crystalline form of the compound of Formula II.

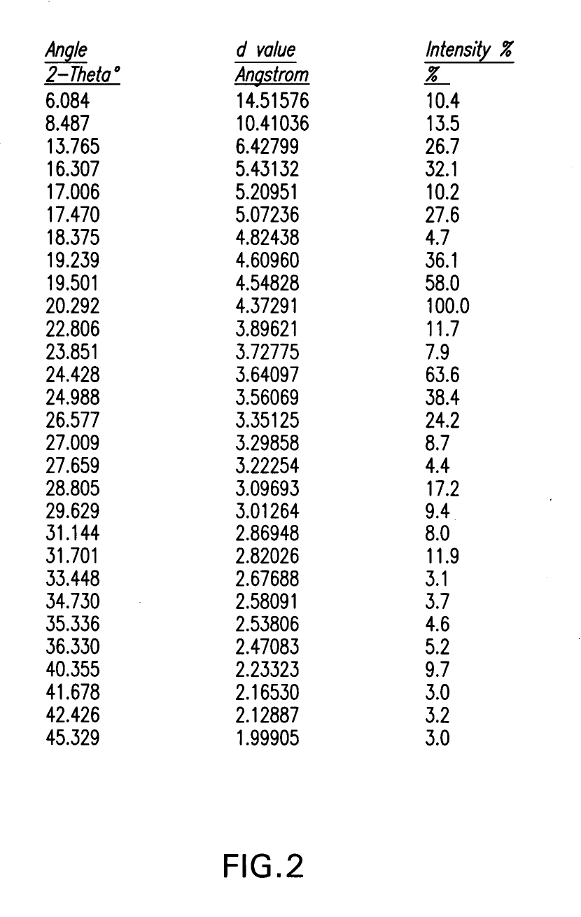

### DETAILED DESCRIPTION OF THE INVENTION

As used herein, in any embodiment of the invention, the term “ambient temperature” refers to a temperature of about 20° C. to about 35° C., preferably about 20° C. to about 25° C.

As used herein, in any embodiment of the invention, the term “chiral auxiliary” refers to a chemical compound or unit that is temporarily incorporated into an organic synthesis so that it can be carried out asymmetrically with the selective formation of one of two enantiomers. Chiral auxiliaries are optically active compounds and introduce chirality in otherwise racemic compounds.

As used herein, in any embodiment of the invention, the term “alkyl” refers to a straight or branched hydrocarbon chain radical consisting of carbon and hydrogen atoms, containing no unsaturation, having from one to eight, preferably one to six, and more preferably one to four carbon atoms, and which is attached to the rest of the molecule by a single bond, e.g., methyl, ethyl, n-propyl, 1-methylethyl (isopropyl), n-butyl, n-pentyl, 1,1-dimethylethyl (t-butyl), or the like.

As used herein, in any embodiment of the invention, the term “aryl” refers to aromatic radicals having in the range of 6 up to 14, preferably 6 to 12, and more preferably 6 to 10 carbon atoms, such as phenyl, substituted phenyl, naphthyl, tetrahydronapthyl, indanyl, biphenyl and the like.

As used herein, in any embodiment of the invention, the term “arylalkyl” refers to an aryl group as defined above directly bonded to an alkyl group as defined above. e.g., —CH₂C₆H₅ (benzyl), —C₂H₄C₆H₅ (ethyl phenyl), and the like, and preferably having 7 to 15, more preferably 7 to 12 carbon atoms.

As used herein, in any embodiment of the invention, the term “alkoxy” denotes alkyl group as defined above attached via oxygen linkage to the rest of the molecule. Representative examples of those groups include —OCH₃, —OC₂H₅ and the like, and preferably those having 1 to 8, more preferably 1 to 6 carbon atoms.

As used herein, in any embodiment of the invention, the term “alkoxycarbonyl” denotes —C(O)— is linked to alkoxy group such —C(O)OCH₃, —C(O)OC₂H₅ etc, and preferably those having 2 to 9, more preferably 2 to 6 carbon atoms.

As used herein, in any embodiment of the invention, the term “cycloalkyl” denotes a non-aromatic mono or multicyclic ring system of about 3 to 12, preferably 3 to 9, and more preferably 3 to 6 carbon atoms, such as cyclopropyl, cyclobutyl, cyclopentyl, cyclohexyl and examples of multicyclic cycloalkyl groups include perhydronapththyl (decalin), adamantyl and norbornyl groups bridged cyclic group or sprirobicyclic groups, e.g sprio (4,4) non-2-yl.

In any embodiment of the invention, the term “substituted” (e.g., as used in “substituted” alkyl, alkoxy, aryl, arylalkyl, or alkoxycarbonyl) refers to the replacement of one or more, preferably from one to three, hydrogen atoms with one or more substituents, examples of which include hydroxy, carboxyl, alkyl (e.g., C₁ to C₆), alkoxy (e.g., C₁ to C₆), aryl (e.g., C₆ to C₁₄), arylalkyl (C₇₋₁₅), cycloalkyl (C₃ to C₁₂), amino, or the like. Further, such substituents may include such groups as alkylthio (e.g., C₁ to C₆), nitro, halo, cyano, haloalkyl (e.g., C₁ to C₆), haloalkoxy (e.g., C₁ to C₆), carboxamido, mono(C₁ to C₆ alkyl)amino, di(C₁ to C₆ alkyl)amino, C₁ to C₆ alkylsulfonylamino, or the like. The substituents may be the same or different.

In any embodiment of the invention, a mixed anhydride as in any embodiment of the invention is an anhydride formed by the reaction of the acid and a chloroformate such as isobutyl chloroformate. A preferred mixed anhydride is isobutyl chloroformate.

An activated ester of a carboxylic acid means a O—CO—R (e.g., mixed anhydride), preferably where R is C₁₋₈, C₁₋₆, or C₁₋₄ alkyl, and includes, e.g., p-nitrophenyl, 2,4,5-trichlorophenyl, hydroxybenzotriazole hydrate (HOBT.H₂O), pentafluorophenol, and N-hydroxysuccinimide carboxylate esters.

Unless otherwise specified, substituents denominated in the compounds or processes of the invention are defined as follows:

X is hydrogen or a substituted or unsubstituted alkyl, preferably C₁₋₈ alkyl, more preferably a C₁₋₆ alkyl, and more preferably a C₁₋₄ alkyl, e.g., methyl;

Y is hydrogen or a substituted or unsubstituted alkyl, preferably C₁₋₈ alkyl, more preferably a C₁₋₆ alkyl, and more preferably a C₁₋₄ alkyl, e.g., methyl; preferably, X and Y are not both hydrogen with respect to the compounds of Formula XII, X, IV, and IV;

n is an integer between 0 and 3, preferably 1;

B is O or S;

M is O, S, or NR₂;

D is substituted or unsubstituted alkoxy, preferably a C₁₋₈ alkoxy, preferably a C₁₋₆ alkoxy, and more preferably a C₁₋₄ alkoxy;

D₁ is hydroxyl, or substituted or unsubstituted alkoxy, preferably a C₁₋₈ alkoxy, preferably a C₁₋₆ alkoxy, and more preferably a C₁₋₄ alkoxy;

P is a hydroxyl protecting group;

Z is a halogen, an activated ester of a carboxylic acid, —O—CO—R (mixed anhydride) or —O—COOR;

R₁ is a substituted or unsubstituted alkyl (preferably C₁₋₈, C₁₋₆, or C₁₋₄), aryl (preferably C₆₋₁₄, C₆₋₁₂), arylalkyl (preferably C₇₋₁₅, C₇₋₁₂), or alkoxycarbonyl (preferably C₂₋₉, C₂₋₆);

R₂ is hydrogen or a substituted or unsubstituted alkyl, preferably C₁₋₈ alkyl, C₁₋₆ alkyl, and more preferably a C₁₋₄ alkyl;

R₃ is a substituted or unsubstituted alkyl, preferably C₁₋₈ alkyl, C₁₋₆ alkyl, and more preferably a C₁₋₄ alkyl;

R₄ is a substituted or unsubstituted alkyl, preferably C₁₋₈ alkyl, C₁₋₆ alkyl, and more preferably a C₁₋₄ alkyl;

R₅ is a substituted or unsubstituted alkyl, preferably C₁₋₈ alkyl, C₁₋₆ alkyl, and more preferably a C₁₋₄ alkyl; and

R₆ is a substituted or unsubstituted alkyl, preferably C₁₋₈ alkyl, C₁₋₆ alkyl, and more preferably a C₁₋₄ alkyl.

In one embodiment, the present invention encompasses a compound of Formula XII: 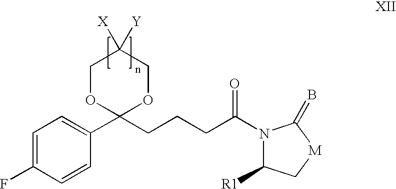
wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl, with the proviso that X and Y are not both hydrogen.

Preferably, n is 0-2; B and M are O or S; and R₁ is C₆-C₁₄ aryl. Preferably, R₁ is selected from the group consisting of: phenyl, naphthyl, tetrahydronapthyl, indanyl, and biphenyl. Also preferably, X and Y are methyl; n is 1; B and M are O; and R₁ is phenyl.

In another embodiment, the invention encompasses a process for preparing the compound of Formula XII comprising combining the compound of Formula XIII: 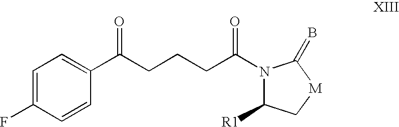
with a diol derivative of Formula III: 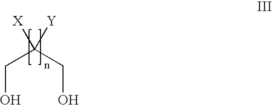
to obtain the compound of Formula XII, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl.

Preferably, X and Y for the compound of Formula XII are not both hydrogen. Preferably, B is O. Preferably, X and Y are methyl. Preferably, n is 1.

Preferably, the compound of Formula XIII is combined with the diol derivative of Formula III in the presence of at least one of at least one of pyridinum hydrobromide, pyridinum hydrochloride, pyridinum hydroiodide, pyridinum paratoluenesulfonate, pyridinum methane sulfonate, pyridinum benzene sulfonate, pyridinum C₁₋₄ tri-alkyl amine hydrochloride, e.g., pyridinum tri-methyl amine hydrochloride, pyridinum C₁₋₄ tri-alkyl amine hydrobromide, e.g., pyridinum tri-methyl amine hydrobromide, pyridinum C₁₋₄ tri-alkyl amine hydroiodide, e.g., pyridinum tri-methyl amine hydroiodide, C₁₋₄ tri-alkyl silyl chloride, e.g., tri-methyl silyl chloride, BF₃-etherate, para toluene sulfonic acid, benzene sulfonic acid, methane sulfonic acid, sulfonic acid, titanium tetrachloride, titanium tetra isopropoxide, aluminum chloride, or zinc chloride.

The reaction may be neat or in at least one organic solvent. If an organic solvent is used, the organic solvent is preferably selected from the group consisting of: halogenated hydrocarbons, aromatic hydrocarbons, aliphatic cyclic hydrocarbons, or ethers, e.g., a C₁ to C₈ halogenated hydrocarbon, a C₃ to C₁₂ aliphatic cyclic hydrocarbon a C₆ to C₁₄ aromatic hydrocarbon, or a C₃ to C₇ ether. Halogenated hydrocarbons may include cyclic or acyclic, saturated or unsaturated aliphatic or aromatic hydrocarbons. Optionally, the halogenated hydrocarbon is halogenated alkane. Preferably, the halogenated alkane is selected from the group consisting of: chloromethane, dichloromethane, chloroethane, dichlorotrifluoroethane, difluoroethane, hexachloroethane, and pentafluoroethane. Optionally, the halogenated hydrocarbon is halogenated alkene. Preferably, the halogenated alkene is selected from the group consisting of: tetrachloroethene, dichloroethene, trichloroethene, vinyl chloride, chloro-1,3-butadiene, and chlorotrifluoroethylene. Optionally, the halogenated hydrocarbon is halogenated benzene. Preferably, the halogenated benzene is selected from the group consisting of: benzotrichloride, benzyl chloride, bromobenzene, chlorobenzene, chlorotoluene, dichlorobenzene, fluorobenzene, and trichlorobenzene. Preferably, the halogen is chlorine. More preferably, the halogenated hydrocarbon is halogenated aromatic hydrocarbon or halogenated C₁-C₄ alkane. More preferably, the halogenated hydrocarbon is chlorinated aromatic hydrocarbon or chlorinated C₁-C₄ alkanes. More preferably, the halogenated hydrocarbon is selected from the group consisting of: chlorobenzene, o- or p-dichlorobenzene, dichloroethane, dichloromethane, and o-chlorotoluene. Most preferably, the halogenated hydrocarbon is selected from the group consisting of: dichloromethane and dichloroethane. Preferably, the aromatic hydrocarbon is C₅-C₁₄ aromatic hydrocarbon. Preferably, the aromatic hydrocarbon is selected from the group consisting of: toluene and xylene. Preferably, the aliphatic cyclic hydrocarbon is C₃-C₈ aliphatic cyclic hydrocarbon. Preferably, the aliphatic cyclic hydrocarbons selected from the group consisting of: cyclohexane and cyclopentane.

Preferably, the reaction further comprises heating after the compound of Formula XIII is combined with the diol derivative of Formula III, more preferably to a temperature of about ambient temperature to about reflux temperature, preferably about 40° C. to about 110° C., and more preferably about 80° C. to about 110° C.

The present invention encompasses a process for preparing the compound of Formula XII comprising combining a compound of Formula VIII: 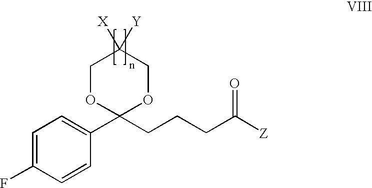
with a chiral auxiliary of Formula IX: 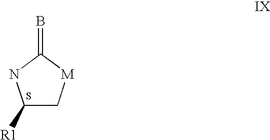
to form the compound of Formula XII, as illustrated in the following scheme: 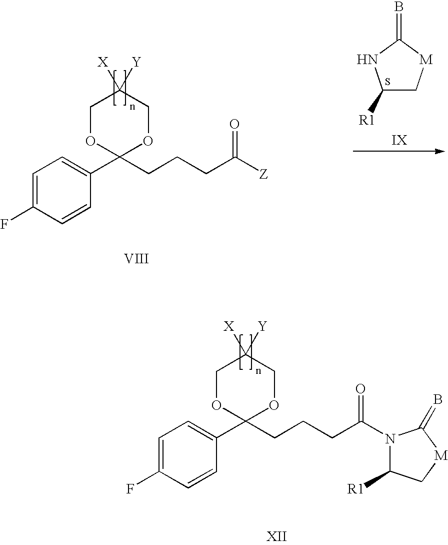
wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl; Z is a halogen, an activated ester of a carboxylic acid, —O—CO—R or —O—COOR; and R is C₁₋₈ alkyl.

Preferably, X and Y for the compound of Formula XII are not both hydrogen. Preferably, X and Y are methyl. Preferably, n is 0, 1, or 2. More preferably, n is 1. Preferably, R is selected from the group consisting of: methyl, ethyl, isobutyl, and tertiary butyl. Preferably, Z is Cl or OCOC(CH₃)₃; and more preferably OCOC(CH₃)₃. Preferably, B is O or S, and more preferably O.

Preferably, M is O or S, and more preferably is O. Preferably R₁ is aryl, and more preferably phenyl. Preferably, R₂ is hydrogen or alkyl, and more preferably methyl.

In another embodiment, the invention encompasses a process for preparing the compound of Formula XII comprising (a) reacting a 4-fluorobenzoyl butyric acid or ester of Formula V: 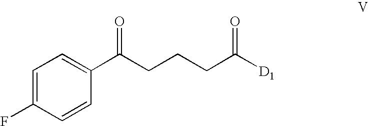
with a diol derivative of Formula III: 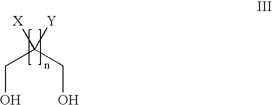
to form a compound of Formula VI: 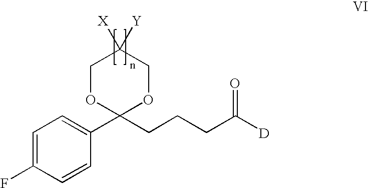
(b) reacting the compound of Formula VI with an inorganic base to obtain a compound of Formula VII: 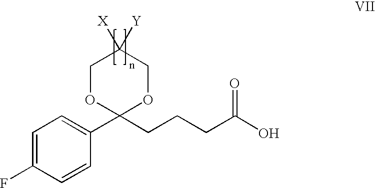
(c) converting the compound of Formula VII to a compound of Formula VIII: 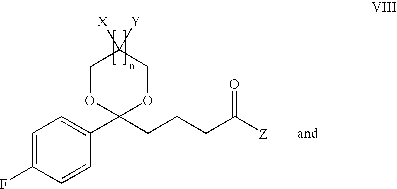
(d) reacting the compound of Formula VIII with a chiral auxiliary of Formula IX: 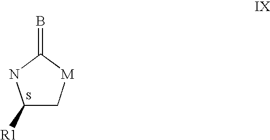
to obtain a compound of Formula XII, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl; Z is a halogen, an activated ester of a carboxylic acid, —O—CO—R or —O—COOR; and R is C₁₋₈ alkyl; D is substituted or unsubstituted C₁₋₈ alkoxy, and D₁ is hydroxyl, or substituted or unsubstituted C₁₋₈ alkoxy. Preferably, X and Y for the compound of Formula XII are not both hydrogen.

The process is illustrated in the following scheme:

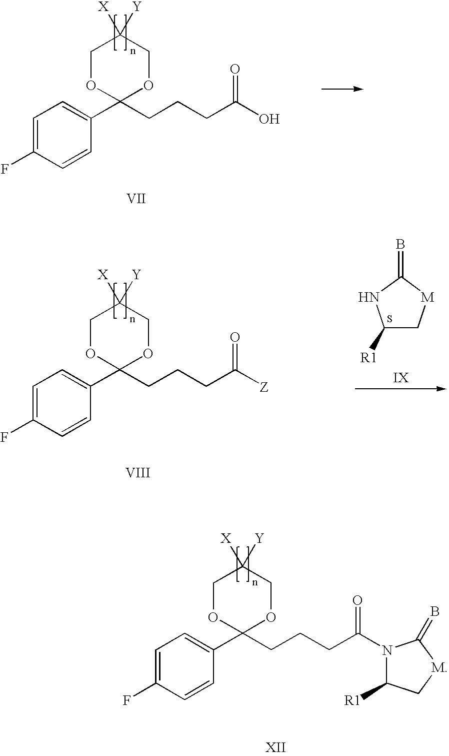

Preferably, the 4-fluorobenzoyl butyric acid or ester of formula V is reacted with the diol derivative of Formula III in the presence of at least one of pyridinium hydrobromide, pyridinium paratoluenesulfonate, pyridinium methane sulfonate, pyridinium benzene sulfonate, triethyl amine hydrochloride, trimethyl silyl chloride, BF₃-etherate, para toluene sulfonic acid, benzene sulfonic acid, methane sulfonic acid, or sulfonic acid to form the compound of Formula VI.

In another embodiment, the present invention encompasses a process for preparing ezetimibe by preparing the compound of Formula XII as described above, preferably where X and Y are not both hydrogen, and converting it to ezetimibe.

In one embodiment, the invention encompasses a compound of Formula VI: 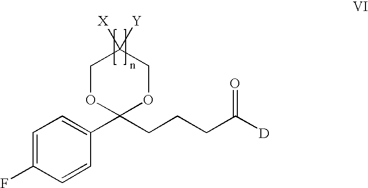
wherein X is hydrogen or a substituted or unsubstituted C₁₋₈ alkyl; Y is hydrogen or a substituted or unsubstituted C₁₋₈ alkyl; n is an integer between 0 and 3; and D is substituted or unsubstituted C₁₋₈ alkoxy.

Preferably, X and Y are methyl. Preferably, n is 0, 1 or 2, and more preferably 1. Preferably, the alkoxy is selected from the group consisting of: OCH₃; OC₂H₅ and OCH₂C(CH₃)₂CH₂OH.

In another embodiment, the invention encompasses a process for preparing the compound of Formula VI comprising reacting a 4-fluorobenzoyl butyric acid or ester of Formula V: 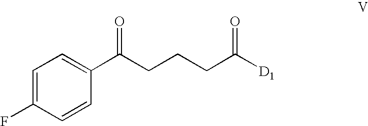
wherein D₁ is hydroxyl, substituted or unsubstituted C₁₋₈ alkoxy, with a diol derivative of Formula III: 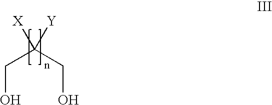
to obtain a compound of Formula VI. The process is illustrated in the following scheme:

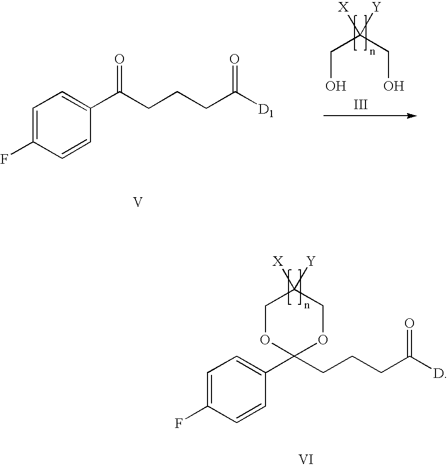

Preferably, the substitutions on the alkoxy group are substituted or unsubstituted alkyl, hydroxyl substituted alkyl. Preferably, the 4-fluorobenzoyl butyric acid or ester of Formula V is reacted with the diol derivative of Formula III in the presence of Py.HBr, PPTS, or p-TSA.

Preferably, the 4-fluorobenzoyl butyric acid or ester of formula V is reacted with the diol derivative of Formula III in the presence of at least one of pyridinium hydrobromide, pyridinium paratoluenesulfonate, pyridinium methane sulfonate, pyridinium benzene sulfonate, triethyl amine hydrochloride, trimethyl silyl chloride, BF₃-etherate, para toluene sulfonic acid, benzene sulfonic acid, methane sulfonic acid, or sulfonic acid to form the compound of Formula VI.

The present invention also encompasses a process for preparing azetidinone by preparing the compound of Formula VI as described above, and converting it to azetidinone, particularly ezetimibe.

The present invention also encompasses a compound of Formula VII: 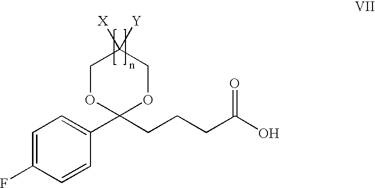
wherein X is hydrogen or a substituted or unsubstituted C₁₋₈ alkyl; Y is hydrogen or a substituted or unsubstituted C₁₋₈ alkyl; and n is an integer between 0 and 3.

Preferably, X and Y are methyl. Preferably, n is selected from 0, 1 and 2, and more preferably, n is 1.

In another embodiment, the invention encompasses a process for preparing the compound of Formula VII comprising reacting a compound of Formula VI: 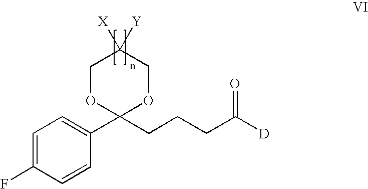
with an inorganic base to obtain the compound of Formula VII, wherein
D is substituted or unsubstituted C₁₋₈ alkoxy. The process is illustrated in the following scheme:

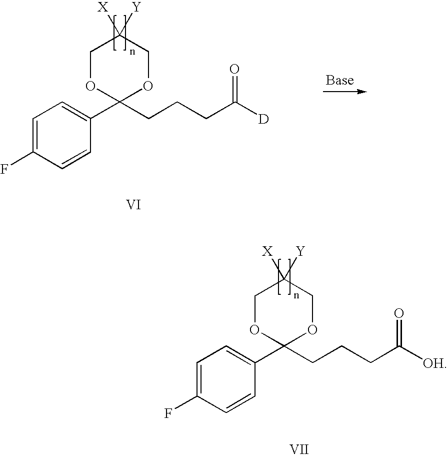

Preferably, the inorganic base is at least one selected from the group consisting of alkali metal carbonate, alkali metal bicarbonate, and alkali metal hydroxide. Preferred alkali metal carbonates include sodium carbonate and potassium carbonate. Preferred alkali metal bicarbonates include sodium bicarbonate and potassium bicarbonate. Preferred alkali metal hydroxides include sodium hydroxide and potassium hydroxide.

The present invention encompasses a process for preparing ezetimibe comprising preparing the compound of Formula VII as described above, and converting it to azetidinone, particularly ezetimibe.

The present invention also encompasses a compound of Formula VIII: 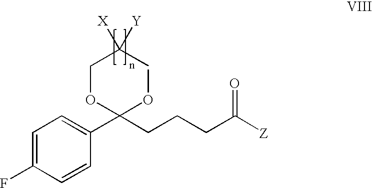
wherein X is hydrogen or a substituted or unsubstituted C₁₋₈ alkyl; Y is hydrogen or a substituted or unsubstituted C₁₋₈ alkyl; n is an integer between 0 and 3; Z is a halogen, an activated ester of a carboxylic acid, —O—CO—R or —O—COOR; and R is C₁₋₈ alkyl.

Preferably, X and Y are methyl. Preferably, R is methyl, ethyl, isobutyl, or tertiary butyl. Preferably, Z is Cl or OCOC(CH₃)₃, more preferably, Z is OCOC(CH₃)₃. Preferably, the activated ester of a carboxylic acid is selected from the group consisting of: hydroxybenzotriazole hydrate (HOBT.H2O), pentafluorophenol, and N-hydroxysuccinimide carboxylate ester.

In another embodiment, the present invention encompasses a process for preparing the compound of Formula VIII comprising reacting a compound of Formula VII: 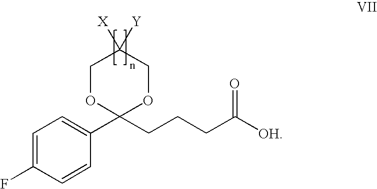
with a at least one reagent selected from the group consisting of thionyl chloride, thionyl bromide, oxalyl chloride, oxalyl bromide, phosphorous penta chloride, phosphorus penta bromide, phosphorous oxychloride, chloroformates, pivolyl chloride, pivolyl bromide, dicyclohexylcarbodiimide (DCC), N-hydroxy succinimde, and 1-hydroxybenzotriazole (HOBT) to form the compound of Formula VIII, as illustrated in the following scheme:

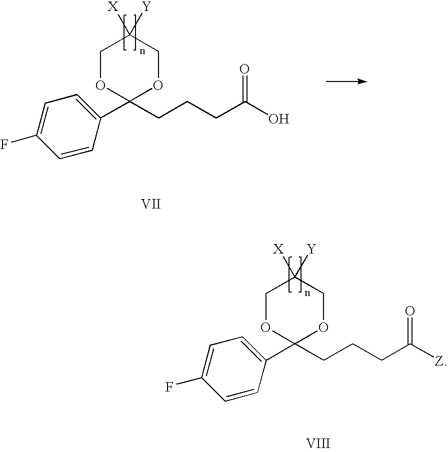

Preferably, X and Y are methyl. Preferably, R is methyl, ethyl, isobutyl, or tertiary butyl. Preferably, Z is Cl or OCOC(CH₃)₃, more preferably, Z is OCOC(CH₃)₃. Preferably, the activated ester of a carboxylic acid is selected from the group consisting of: hydroxybenzotriazole hydrate (HOBT.H₂O), pentafluorophenol, and N-hydroxysuccinimide carboxylate ester.

The present invention also encompasses a process for preparing ezetimibe comprising preparing the compound of Formula VIII as described above, and converting it azetidinone, particularly ezetimibe.

In another embodiment, the present invention encompasses a compound of Formula X: 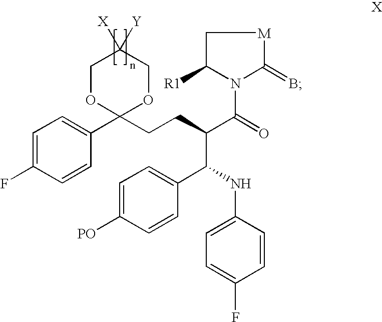
wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; and P is a hydroxyl protecting group, with the proviso that X and Y are not both hydrogen.

Preferably, X and Y are alkyl; n is 0-2; B and M are O or S, R₁ is aryl; and P is hydroxyl protecting group. More preferably, X and Y are methyl; n is 1; B and M are O, R₁ is phenyl, and P is benzyl.

A suitable protecting group, or “P”, for hydroxy functionalities, include acyl, tetrahydropyranyl, tetrahydrothiopyranyl, tetrahydrofuranyl, tetrahydrothiofuranyl, 2-(phenylselenyl)ethyl, o-nitrobenzyl, benzyl, p-methoxy benzyl, tri(C₁₋₈ alkyl)silyl (preferably tri(C₁₋₆ alkyl)silyl) or tri(C₁₋₄ alkyl)silyl, where the (C₁₋₈ alkyl) groups may be the same or different), e.g., trimethylsilyl, triisopropylsilyl, isopropyldimethylsilyl, and t-butyldimethylsilyl, and tri(C₆₋₁₀ aryl)silyl (where the (C₆₋₁₀ aryl) groups may be the same or different), e.g., t-butyldiphenylsilyl, tribenzylsilyl, acetyl, isobutyl, pivaloyl, adamantoyl, benzoyl, 2,4,6-trimethylbenzoly (mesitoyl), methyl carbonyl, p-nitrophenyl carbonyl, p-nitrobenzyl carbonyl, S-benzyl thiocarbonyl, and N-phenylcarboyl. A preferred protecting group is where P is selected from the group consisting of: benzyl, trimethylsilyl, para-methoxy benzyl, acetyl, and methyl. A more preferred protecting group is benzyl.

In another embodiment, the present invention encompasses a process for preparing the compound of Formula X comprising (a) reacting a compound of Formula XII: 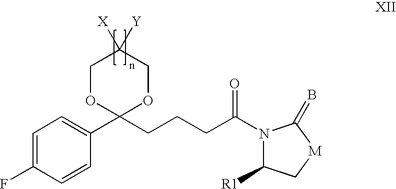
with a compound of Formula XI: 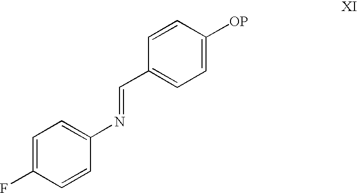
in the presence of a tertiary organic base and at least one acid catalyst selected from the group consisting of para-toluene sulfonic acid, methane sulfonic acid, benzene sulfonic acid, trimethylsilyl chloride (TMSCl), titanium tetrachloride, titanium tetra isopropoxide, aluminum chloride, zinc chloride, and BF₃-etherate to form the compound of Formula X, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; and P is a hydroxyl protecting group. Preferably, X and Y for the compound of Formula XII and the compound of Formula X are not both hydrogen.

Preferably, the compound of Formula XII is reacted with the compound of Formula XI in the presence of at least one organic solvent selected from the group consisting of an aromatic hydrocarbon, a halogenated hydrocarbon, and an ether. Preferred organic solvents are as described above.

Preferably, the tertiary organic base is a tertiary amine, preferably N,N′-Diisopropylethylamine (DIPEA), triethyl amine, or pyridine.

Preferably, the compound of Formula XII is reacted with the compound of Formula XI at a temperature of about −50° C. to about ambient temperature, and more preferably at a temperature of about −30° C. to about 0° C.

Preferably, X and Y are methyl. Preferably, n is an integer between 0 and 2. Preferably, B is O. Preferably, M is O or S. More preferably, M is O. Preferably, the substituted or unsubstituted C₆-C₁₄ aryl is aryl. Preferably, the aryl is phenyl. Preferably, R₂ is hydrogen or alkyl. Preferably, the alkyl is methyl. Most preferably, R₂ is methyl. Preferably, the protecting group is selected from the group consisting of: aryl alkyl, para-methoxybenzyl, and substituted silyl. More preferably, the protecting group is selected from the group consisting of: benzyl, para-methoxybenzyl, and trimethyl silyl.

The present invention also encompasses a process for preparing azetidinone comprising preparing the compound of Formula X as described above, preferably where X and Y for the compound of Formula XII and the compound of Formula X are not both hydrogen, and converting it to azetidinone, particularly ezetimibe.

In one embodiment, the present invention encompasses a compound of Formula IV: 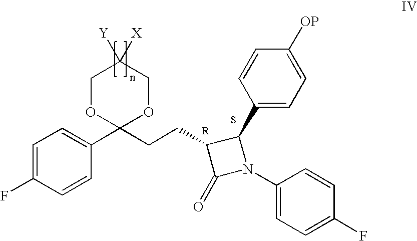
wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group, with the proviso that X and Y are not both hydrogen.

Preferably, X and Y are methyl. Preferably, n is an integer between 0 and 2, more preferably n is 1. Preferably, the hydroxyl protecting group is selected from the group consisting of: aryl alkyl and substituted silyl. More preferably, the protecting group is selected from the group consisting of: benzyl, para-methoxybenzyl, and trimethyl silyl.

In another embodiment, the invention encompasses a process for preparing the compound of Formula IV comprising cyclizing a compound of Formula X: 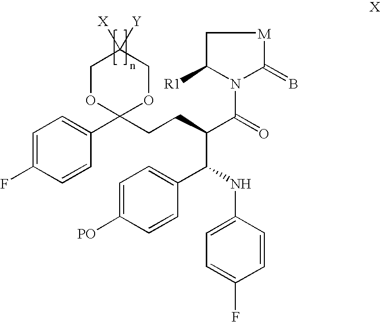
with a silylating agent selected from the group consisting of bis(trimethylsilyl)acetamide (BSA), N-methyl-O-trimethylsilyl acetamide, iso-propenyloxy trimethylsilane, bis(trimethylsilyl)urea (BSU), hexamethyldisilazane (HMDS), and a mixture of hexamethyldisilazane (HMDS) and trimethyl silyl chloride, optionally in the presence of a fluoride ion catalyst, to form the compound of Formula IV, wherein X is hydrogen or a substituted or unsubstituted C₁₋₈ alkyl; Y is hydrogen or a substituted or unsubstituted C₁₋₈ alkyl; n is an integer between 0 and 3; P is a hydroxyl protecting group; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; and R₂ is hydrogen or a substituted or unsubstituted C₁₋₈ alkyl. Preferably, X and Y for the compound of Formula X and the compound of Formula IV are not both hydrogen.

Preferably, the silylating agent is BSA. Preferably, the process further comprises adding a fluoride ion catalyst, more preferably a fluoride ion catalyst of Formula XVII: 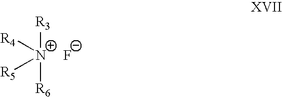
wherein R₃ is a substituted or unsubstituted alkyl (preferably C₁₋₈, C₁₋₆, or C₁₋₄ alkyl) or aryl (preferably C₆₋₁₄, C₆₋₁₂, or C₆₋₁₀ aryl); R₄ is a substituted or unsubstituted alkyl (preferably C₁₋₈, C₁₋₆, or C₁₋₄ alkyl) or aryl (preferably C₆₋₁₄, C₆₋₁₂, or C₆₋₁₀ aryl); R₅ is a substituted or unsubstituted alkyl (preferably C₁₋₈, C₁₋₆, or C₁₋₄ alkyl) or aryl (preferably C₆₋₁₄, C₆₋₁₂, or C₆₋₁₀ aryl); and R₆ is a substituted or unsubstituted alkyl (preferably C₁₋₈, C₁₋₆, or C₁₋₄ alkyl) or aryl (preferably C₆₋₁₄, C₆₋₁₂, or C₆₋₁₀ aryl). A preferred fluoride ion catalyst is tetrabutyl ammonium fluoride (TBAF).

The present invention also encompasses a process for preparing azetidinone by preparing the compound of Formula IV as described above, preferably where X and Y for the compound of Formula X and the compound of Formula IV are not both hydrogen, and converting it azetidinone, particularly ezetimibe.

In one embodiment, the invention encompasses a compound of Formula XV: 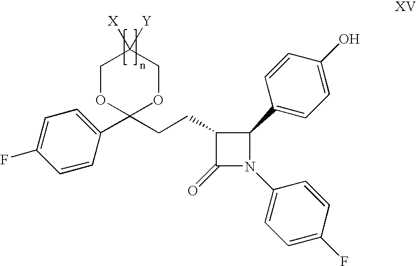
wherein X is hydrogen or a substituted or unsubstituted C₁₋₈ alkyl; Y is hydrogen or a substituted or unsubstituted C₁₋₈ alkyl; and n is an integer between 0 and 3, with the proviso that X and Y are not both hydrogen.

Preferably X and Y are alkyl. Preferably, the alkyl is methyl. Preferably, n is selected from 0, 1 and 2, more preferably, n is 1.

In another embodiment, the invention encompasses a process for preparing the compound of Formula XV comprising converting a compound of Formula IV: 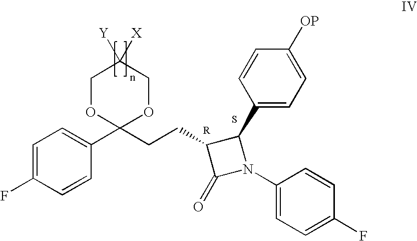
to the compound of Formula XV by removing P using catalytic hydrogenation or hydride transfer reaction using conventional methods known in the literature, or by reacting with quaternary ammonium fluoride in the presence of an organic solvent, wherein P is a hydroxyl protecting group; X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula XV and the compound of Formula IV are not both hydrogen. The process is illustrated below:

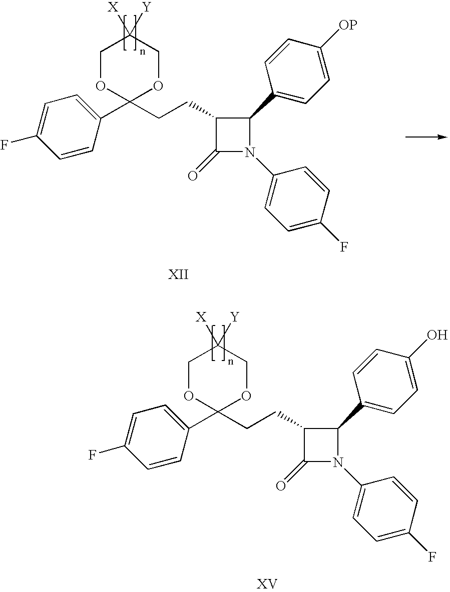

Preferably, the protecting group is an aryl alkyl such as benzyl and para-methoxybenzyl, or a substituted silyl such as trimethyl silyl. More preferable protecting groups include benzyl, para-methoxy benzyl, and trimethyl silyl.

Preferably, the catalyst for catalytic hydrogenation is selected from the group consisting of PtO₂, Pd/C, Pt/C, Rh/C and Raney-Ni. Preferably, the catalyst for catalytic hydrogenation is Pd/C, Rh/C, and more preferably Pd/C.

The conversion may be neat or in at least one organic solvent. If an organic solvent is used, the organic solvent is preferably selected from the group consisting of aromatic hydrocarbons, alcoholic solvents, esters and ethers. Preferably, the aromatic hydrocarbon solvent is toluene or xylene. Preferably, the alcoholic solvent is methanol, ethanol, or propanol. More preferably, the alcoholic solvent is ethanol. Preferable esters include ethyl acetate and propyl acetate.

The present invention also encompasses a process for preparing azetidinone comprising preparing the compound of Formula XV using a process described above, preferably where X and Y for the compound of Formula XV and the compound of Formula IV are not both hydrogen, and converting it to azetidinone, particularly ezetimibe.

In one embodiment, the invention encompasses a process for preparing a compound of Formula XVI: 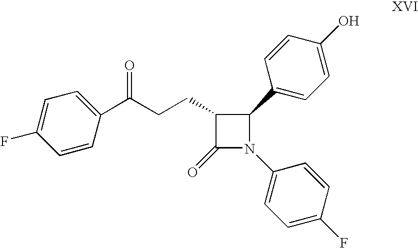
comprising converting a compound of Formula IV: 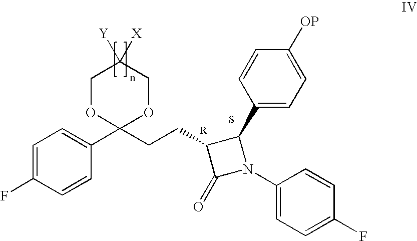
to the compound of Formula XVI using acid hydrolysis, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group; X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula IV are not both hydrogen. The process is illustrated below:

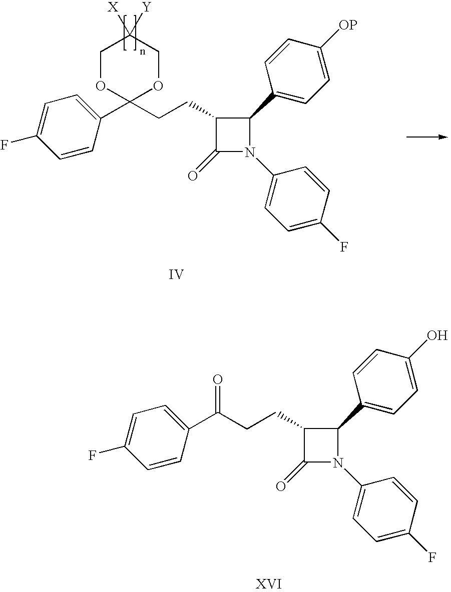

Preferably, X and Y are methyl. Preferably, n is selected from 0, 1 and 2, more preferably, n is 1. Preferably, the protecting group is benzyl, para-methoxybenzyl or trimethyl silyl.

Preferably, the acid used for acid hydrolysis is selected from at least one of formic acid, acetic acid, propionic acid, camphor sulfonic acid, hydrochloric acid, and sulfuric acid. Preferred acids are formic acid, camphor sulfonic acid, and sulfuric acid. More preferable acids include formic acid and sulfuric acid.

The acid hydrolysis may be conducted neat or in at least one organic solvent. If an organic solvent is used, the organic solvent is preferably selected from the group consisting of aromatic hydrocarbons, alcoholic solvents, esters, and ethers. Preferred aromatic hydrocarbon solvents are toluene and xylene, and preferred alcoholic solvents are methanol and ethanol. Preferred esters are propanoate, ethyl acetate, and propyl acetate. Preferred ethers are tetrahydrofuran (THF) and methyl tert-butyl ether (MTBE). A more preferred alcoholic solvent is ethanol.

In another embodiment, the invention encompasses a process for preparing a compound of Formula XVI comprising converting the compound of Formula XV: 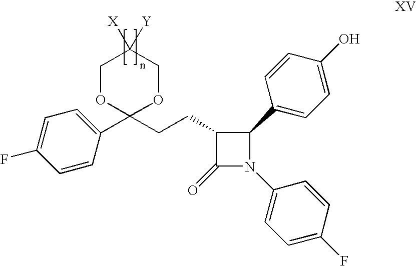
to the compound of Formula XVI in the presence of at least one of an organic acid or a mineral acid, wherein X is hydrogen or a substituted or unsubstituted C₁₋₈ alkyl; Y is hydrogen or a substituted or unsubstituted C₁₋₈ alkyl; and n is an integer between 0 and 3. Preferably, X and Y for the compound of Formula XV are not both hydrogen. The process is illustrated below:

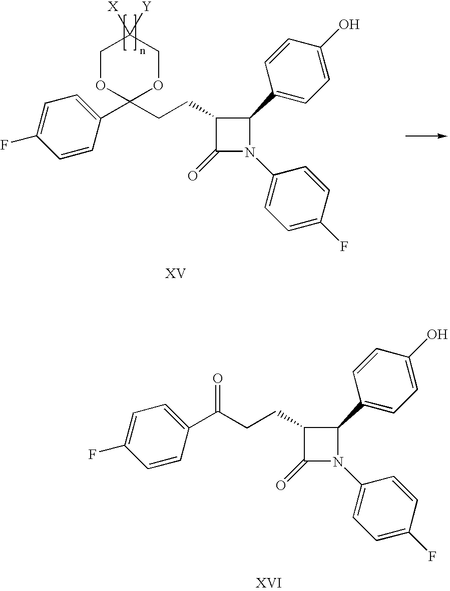

Preferably, X and Y are methyl. Preferably, n is selected from 0, 1 and 2; more preferably, n is 1.

Preferably, the organic acid is at least one selected from the group consisting of: aliphatic acid and aromatic acid. Preferably, the aliphatic acid is selected from the group consisting of: formic acid, acetic acid, camphor sulfonic acid and propionic acid. Preferably, the aromatic acid is selected from the group consisting of: para-toluene sulfonic acid, and benzene sulfonic acid. Preferably, the mineral acid is selected from the group consisting of: e.g., phosphoric acid, hydrobromic acid, hydrochloric acid, or sulfuric acid.

The present invention also encompasses a process for preparing azetidinone comprising preparing the compound of Formula XVI as described above, preferably where X and Y for the compound of Formula XV are not both hydrogen, and converting it to azetidinone, particularly ezetimibe.

In another embodiment, the invention encompasses a process for preparing hydroxyl-alkyl substituted azetidinones, particularly ezetimibe of Formula I: 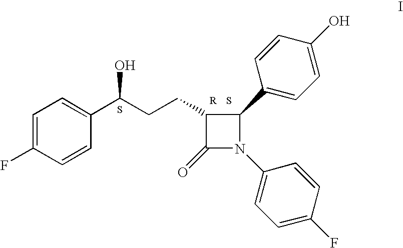
comprising converting a compound of Formula XII, VI, VII, VIII, X, IV, or XV to the azetidinone. Preferably, X and Y for the compounds of Formula XII, IV, X, and XV are not both hydrogen.

The invention also encompasses a novel, simple and high yielding plant friendly process using mild conditions for producing hydroxyl-alkyl substituted azetidinones, particularly ezetimibe useful as hypocholesterolemic agent of Formula I:

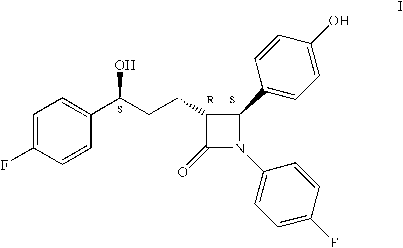

In one embodiment, the invention encompasses a process for preparing hydroxyl-alkyl substituted azetidinones, particularly ezetimibe, comprising at least one of the following steps:
(a) deprotecting a compound of Formula IV: 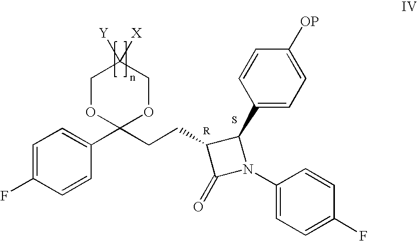
using catalytic hydrogenation, hydride transfer reduction, acid hydrolysis, or reacting with a quaternary ammonium fluoride to obtain a compound of Formula XV: 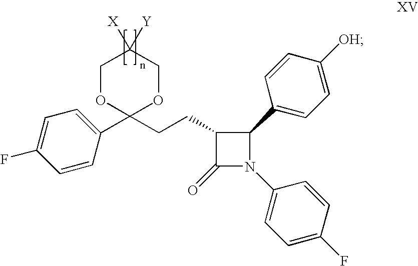
(b) deprotecting the compound of Formula XV with an acid to obtain a compound of Formula XVI: 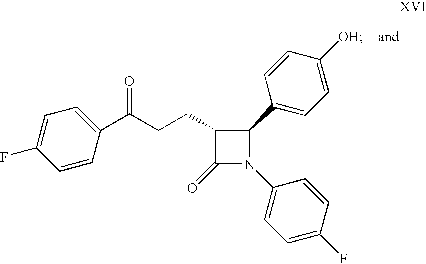
(c) reducing the compound of Formula XVI in the presence of a chiral catalyst to the azetidinone using processes known in the art, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group. Preferably, X and Y for the compounds of Formula IV, and XV are not both hydrogen.

Preferred substituents, reagents, solvents, and/or reaction conditions are as set forth in the processes described above.

In another embodiment, the invention encompasses a process for preparing hydroxyl-alkyl substituted azetidinones, particularly ezetimibe, comprising at least one of the following steps:

(a) reacting a compound of Formula XIII with a diol derivative of Formula III in the presence of at least one acid catalyst selected from the group consisting of para-toluene sulfonic acid, methane sulfonic acid, benzene sulfonic acid, trimethyl silyl chloride, titanium tetrachloride, titanium tetra isopropoxide, aluminum chloride, zinc chloride, BF₃-etherate, or a salt of an organic base selected from the group consisting of pyridinium hydrobromide, pyridinium paratoluenesulfonate, pyridinium methane sulfonate, pyridinium benzene sulfonate, and triethyl amine hydrochloride, neat or in an organic solvent, to obtain a compound of Formula XII, as illustrated in the following scheme:

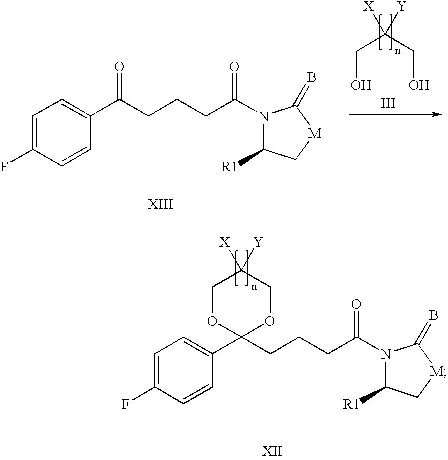

(b) reacting the compound of Formula XII with a compound of Formula XI to obtain a compound of Formula X, preferably at a temperature of about −50° C. to about ambient temperature, as illustrated in the following scheme:

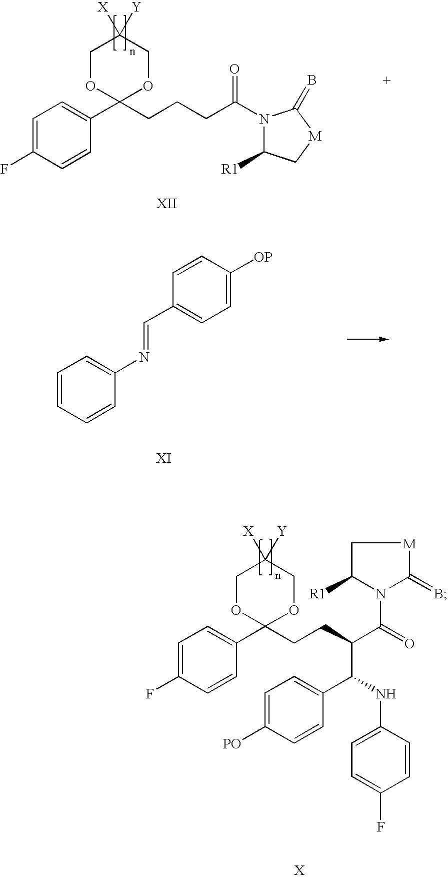

(c) combining the compound of Formula X with a silylating agent and optionally a fluoride ion catalyst of Formula XVII to obtain a compound of Formula IV:

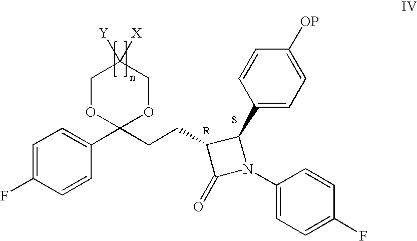

(d) combining the compound of Formula IV with an acid, preferably at least one acid selected from the group consisting of: formic acid, acetic acid, propionic acid, camphor sulfonic acid, hydrochloric acid, and sulfuric acid, to obtain a compound of Formula II:

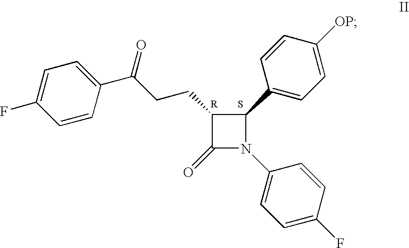

(e) converting the compound of Formula II to the compound of Formula XIV 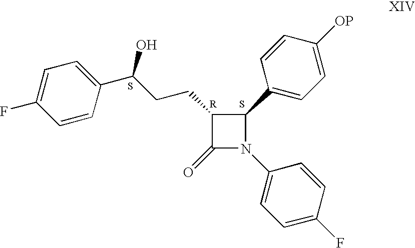
using one of the following methods: (i) combining the compound of Formula II with at least one chiral reducing agent selected from the group consisting of β-chloro diisopinocamphenyl borane, borane, borane-methyl sulfide complex, borane-morpholine complex, borane-pyridine complex, borane-tetrahydrofuran complex, borane-tributylphosphine complex, borane-triethylamine complex, borane-trimethylamine complex, borane-1,4 thioxane, borane-dimethylsulfide complex, borane 1,4-dioxane, borane diethylaniline, borane N-ethyl-N-isopropylaniline, N-borane phenylamine, and catecholborane in the presence of at least one chiral catalyst selected from the group consisting of:
(R)-tetrahydro-1-methyl-3,3-diphenyl-1H,3H-pyrrol(1,2-c)(1,3,2)oxaza-borolideine,
(R)-tetrahydro-1-butyl-3,3-diphenyl-1H,3H-pyrrol(1,2-c)(1,3,2)oxaza-borolideine, and
(R)-tetrahydro-1-phenyl-3,3-diphenyl-1H,3H-pyrrol(1,2-c)(1,3,2)oxaza-borolideine,
   and optionally at least one solvent selected from the group consisting of:
   dichloromethane, tetrahydrofuran, toluene, 2-methyl tetrahydrofuran, ethyl acetate, and tert-butyl methyl ether, to obtain the compound of Formula XIV;

by (ii) combining the compound of Formula II with at least one chiral catalyst selected from the group consisting of [[RuCl₂(p-cymene)]₂, (S,S)-i-Pr—SO₂DPEN], [[RuCl₂(p-cymene)]₂(S,S)-MsDPEN], [[RuCl₂(hexaMe-benzene)]₂(S,S)-MsDPEN], [[RuCl₂(benzene)]₂(S,S)-MsDPEN], [[RuCl₂(p-cymene)]₂(S,S)-MesithylSO₂-DPEN], [[RuCl₂(p-cymene)]₂(S,S)-2-naphthyl-DPEN], [[RuCl₂(p-cymene)]₂(S,S)-n-Butyl-DPEN], [[RuCl₂(mesitylene)]₂(S,S)-Ms-DPEN], [[RuCl₂(mesitylene)]₂(S,S)-iPr—SO₂-DPEN], [[RuCl₂(hexaMe-benzene)]₂(S,S)-iPr—SO₂-DPEN], [[RuCl₂(p-cymene)]₂(S,S)-n-Bu-SO₂-DPEN], [[RuCl₂(mesitylene)]₂(S,S)-n-Bu-SO₂-DPEN], [[RuCl₂(hexaMe-benzene)]₂(S,S)-n-Bu-SO₂-DPEN], [[RuCl₂(p-cymene)]₂(S,S)-Bn-SO₂-DPEN], [[RuCl₂(p-cymene)]₂(S,S)—Cs-DPEN], [[RuCl₂(p-cymene)]₂(S,S)-3,4-diMe-Ph-SO₂-DPEN], [[RuCl₂(p-cymene)]₂(S,S)-1-Naphthyl-DPEN], [[RuCl₂(p-cymene)]₂(S,S)-1,3,5-tri-iPr-Ph-DPEN], [[RuCl₂(p-cymene)]₂(S,S)-3,4-diMeO-PhSO₂-DPEN], [[RuCl₂(mesitylene)]₂(S,S)—CF₃—SO₂-DPEN], [[RuCl₂(p-cymene)]₂(S,S)-pClPh-SO₂-DPEN], [[RuCl₂(p-cymene)]₂(S,S)-pPh-Ph-SO₂-DPEN], [[RUCl₂(p-cymene)]₂(S,S)-pCF₃-Ph-SO₂-DPEN], [[RuCl₂(p-cymene)]₂(S,S)-pNO₂-Ph-SO₂-DPEN], [[RuCl₂(p-cymene)]₂(S,S)-pMeO-Ph-SO₂-DPEN], [[RuCl₂(p-cymene)]₂(S,S)-Ms-DACH], [[RuCl₂(p-cymene)]₂(S,S)-1-Naphtyl-SO₂-DACH], [(S,S)-Tethered —RuCl], [[RhCl₂Cp*]₂(S,S)-Ms-DPEN], [[RhCl₂Cp*]₂(S,S,S)—Cs-DPEN], and [[RuCl₂(mesitylene)]₂(S,S)-i-Bu-SO₂-DPEN]; at least one hydrogen source selected from the group consisting of formic acid or a salt thereof, C₃-C₁₃ secondary alcohol, and cyclohexadiene; and an organic solvent to obtain the compound of Formula XIV; or

by (iii) combining the compound of Formula II with a chiral catalyst under an inert gas environment; adding a base to obtain a reaction mixture; and subjecting the reaction mixture to a hydrogen pressure of about 2 bars to about 40 bars to obtain the compound of Formula XIV; or

using processes known in the art (such as those described in U.S. Pat. Nos. 5,631,365; 6,207,822, or co-pending Application Ser. No. 60/715,919, filed on Sep. 8, 2005, or application filed on Apr. 10, 2007, entitled “Reduction processes for the preparation of ezetimibe,” attorney docket No. 1662/A425P1, each of which is incorporated herein by reference in its entirety), to obtain a compound of Formula XIV:

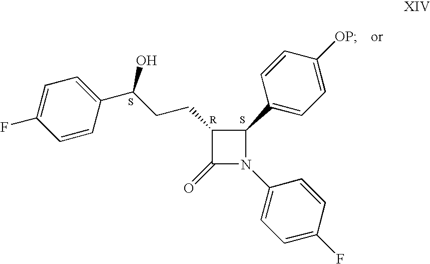

(f) deprotecting group the compound of Formula XIV to obtain the azetidinone, wherein X is hydrogen or a substituted or unsubstituted C₁₋₈ alkyl; Y is hydrogen or a substituted or unsubstituted C₁₋₈ alkyl; n is an integer between 0 and 3; B is O or S; M is O, S, or NR₂; R₁ is a substituted or unsubstituted C₁₋₈ alkyl, C₆₋₁₄ aryl, C₇₋₁₅ arylalkyl, or C₂₋₉ alkoxycarbonyl; R₂ is hydrogen or a substituted or unsubstituted C₁₋₈ alkyl; P is a hydroxyl protecting group; R₃ is a substituted or unsubstituted C₁₋₈ alkyl or C₆₋₁₄ aryl; R₄ is a substituted or unsubstituted C₁₋₈ alkyl or C₆₋₁₄ aryl; R₅ is a substituted or unsubstituted C₁₋₈ alkyl or C₆₋₁₄ aryl; and R₆ is a substituted or unsubstituted C₁₋₈ alkyl or C₆₋₁₄ aryl.

Preferably, X and Y for the compounds of Formula XII, X, and IV are not both hydrogen. Other preferred substituents, reagents, solvents, and/or reaction conditions are as set forth in the processes described above. Preferably, the compound of Formula X is recovered prior to step (c). Preferably, the azetidinone is a compound of Formula I:

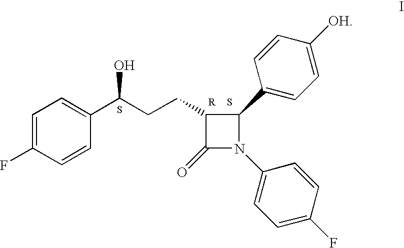

Formula XIV may be converted to the compound of Formula I using processes known in the art (such as those described in U.S. Pat. No. 5,631,365 or 6,207,822). For example, Formula XIV may be converted to the compound of Formula I by adding 10% Pd/C to a solution of 3R,4S)-4-(4-benzyloxyphenyl)-1(4-fluorophenyl)-3-(3(S)-3-(4-Fluorophenyl)-3-hydroxypropyl)-2-azetidinone in ethanol at 20-25° C., stirring for 4 hours under H₂ pressure (60 psi) at 30-35° C. to obtain a residue. The obtained residue may be further crystallized in aqueous isopropanol (“IPA”) to obtain 1-(4-fluorophenyl)-3(R)-[3-(4-fluorophenyl)-3(S)-hydroxypropyl]-4(S)-(4-hydroxyphenyl)-2-azetidinone (ezetimibe).

In another embodiment, the invention encompasses a process for preparing a compound of Formula II:

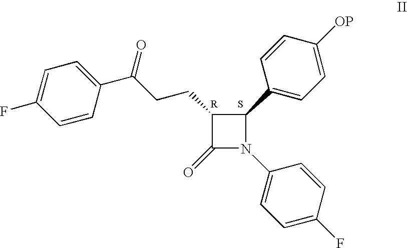

comprising combining a compound of Formula IV: 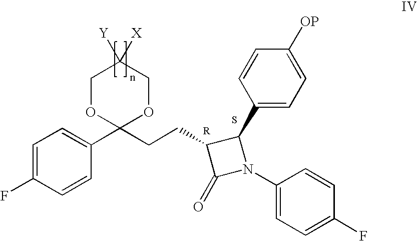
with an acid to, preferably at least one of formic acid, acetic acid, propionic acid, camphor sulfonic acid, hydrochloric acid, or sulfuric acid, obtain the compound of Formula II, wherein X is hydrogen or a substituted or unsubstituted C₁₋₈ alkyl; Y is hydrogen or a substituted or unsubstituted C₁₋₈ alkyl; n is an integer between 0 and 3; and P is a hydroxyl protecting group. Preferably, X and Y for the compound of Formula IV are not both hydrogen.

In another embodiment, the invention encompasses a process for preparing a compound of Formula XIV: 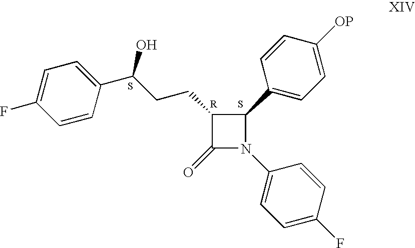
comprising combining a compound of Formula II 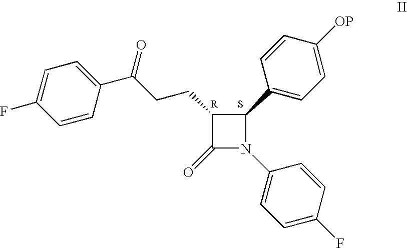
with at least one chiral catalyst selected from the group consisting of [[RuCl₂(p-cymene)]₂, (S,S)-i-Pr—SO₂DPEN], [[RuCl₂(p-cymene)]₂(S,S)-MsDPEN], [[RuCl₂(hexaMe-benzene)]₂(S,S)-MsDPEN], [[RuCl₂(benzene)]₂(S,S)-MsDPEN], [[RuCl₂(p-cymene)]₂(S,S)-MesithylSO₂-DPEN], [[RuCl₂(p-cymene)]₂(S,S)-2-naphthyl-DPEN], [[RuCl₂(p-cymene)]₂(S,S)-n-Butyl-DPEN], [[RuCl₂(mesitylene)]₂(S,S)-Ms-DPEN], [[RuCl₂(mesitylene)]₂(S,S)-iPr—SO₂-DPEN], [[RuCl₂(hexaMe-benzene)]₂(S,S)-iPr—SO₂-DPEN], [[RuCl₂(p-cymene)]₂(S,S)-n-Bu-SO₂-DPEN], [[RuCl₂(mesitylene)]₂(S,S)-n-Bu-SO₂-DPEN], [[RuCl₂(hexaMe-benzene)]₂(S,S)-n-Bu-SO₂-DPEN], [[RuCl₂(p-cymene)]₂(S,S)-Bn-SO₂-DPEN], [[RuCl₂(p-cymene)]2(S,S)—Cs-DPEN], [[RuCl₂(p-cymene)]₂(S,S)-3,4-diMe-Ph-SO₂-DPEN], [[RuCl₂(p-cymene)]₂(S,S)-1-Naphthyl-DPEN], [[RuCl₂(p-cymene)]₂(S,S)-1,3,5-tri-iPr-Ph-DPEN], [[RuCl₂(p-cymene)]₂(S,S)-3,4-diMeO-PhSO₂-DPEN], [[RuCl₂(mesitylene)]₂(S,S)—CF₃—SO₂-DPEN], [[RuCl₂(p-cymene)]₂(S,S)-pClPh-SO₂-DPEN], [[RuCl₂(p-cymene)]₂(S,S)-pPh-Ph-SO₂-DPEN], [[RuCl₂(p-cymene)]₂(S,S)-pCF₃-Ph-SO₂-DPEN], [[RuCl₂(p-cymene)]₂(S,S)-pNO₂-Ph-SO₂-DPEN], [[RuCl₂(p-cymene)]₂(S,S)-pMeO-Ph-SO₂-DPEN], [[RuCl₂(p-cymene)]₂(S,S)-Ms-DACH], [[RuCl₂(p-cymene)]₂(S,S)— 1-Naphtyl-SO₂-DACH], [(S,S)-Tethered-RuCl], [[RhCl₂Cp*]₂(S,S)-Ms-DPEN], [[RhCl₂Cp*]₂(S,S,S)—Cs-DPEN], and [[RuCl₂(mesitylene)]₂(S,S)-i-Bu-SO₂-DPEN]; at least one hydrogen source selected from the group consisting of formic acid or a salt thereof, C₃-C₁₃ secondary alcohol, and cyclohexadiene; and an organic solvent to obtain the compound of Formula XIV, wherein P is a hydroxyl protecting group.

Preferably, the hydroxyl protecting group is selected from the group consisting of benzyl and silyl, e.g., (R^{a})(R^{b})(R^{c})—Si—, wherein R^{a}, R^{b} and R^{c} are the same or different and each are selected from the group consisting of C₁ to C₆ alkyl, phenyl, benzyl, or the like. Preferably, the silyl protecting group is selected from trimethylsilyl or tert-butyldimethylsilyl. Preferably, P is benzyl.

Preferably, the hydrogen source is formic acid or a salt thereof or isopropanol, and more preferably formic acid or a salt thereof. Preferably, the chiral catalyst is [[RuCl₂(p-cymene)]₂(S,S)-i-Pr—SO₂DPEN], [[RuCl₂(mesitylene)]₂(S,S)-iPr—SO₂-DPEN], [[RuCl₂(hexaMe-benzene)]₂(S,S)-n-Bu-SO₂-DPEN], [[RuCl₂(p-cymene)]₂(S,S)—Cs-DPEN], [[RhCl₂Cp*]₂(S,S)-Ms-DPEN], or [RhCl₂Cp*]₂(S,S,S)—Cs-DPEN].

Preferably, the organic solvent is selected from the group consisting of dichloroethane, dichloromethane, methyl tert butyl ether, ethyl acetate, toluene, and mixtures thereof. Methyl tert butyl ether, ethyl acetate, and toluene are preferred, and methyl tert butyl ether are more preferred. Preferably, the process further comprises adding an organic base, preferably triethylamine.

In another embodiment, the invention encompasses a process for preparing a compound of Formula XIV: 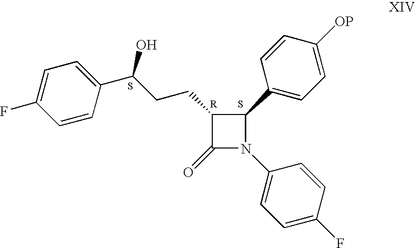
comprising combining a compound of Formula II 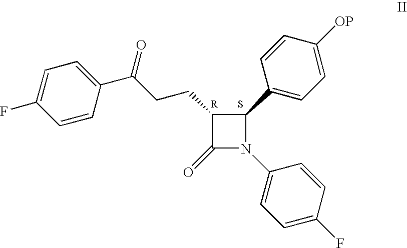
with [(R)-XylPPhosRuCl₂(R)-DAIPEN]; adding a base to obtain a reaction mixture; and subjecting the reaction mixture to a hydrogen pressure of about 2 bars to about 40 bars to obtain the compound of Formula XIV, wherein P is a hydroxyl protecting group, preferably benzyl.

Preferably, the hydrogen pressure is about 4 bars to about 30 bars, and more preferably 25 bars. Preferably, the base is an inorganic base, e.g., tert-butoxide.

In another embodiment, the invention encompasses the use of a diol derivative of Formula III:

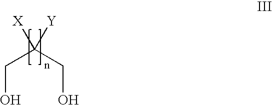

as a carbonyl protecting group for the manufacture of ezetimibe, wherein X is hydrogen or a substituted or unsubstituted C₁₋₈ alkyl; Y is hydrogen or a substituted or unsubstituted C₁₋₈ alkyl; and n is an integer between 0 and 3.

The present invention also provides a novel crystalline compound of Formula II, i.e., 3-{4-[2-(4-Fluorophenyl)-5,5-dimethyl-[1,3]dioxan-2-yl]butyryl}-(4S)-phenyl-1,3-oxazolidin-2-one. In one embodiment, the crystalline form is characterized by a PXRD pattern having peaks at about 16.3, 19.5, 20.3, 24.4, and 25.0±0.2 degrees two-theta. Preferably, the crystalline form is further characterized by a PXRD pattern having peaks at about 13.8, 17.5, 26.6, and 28.8 degrees two-theta.

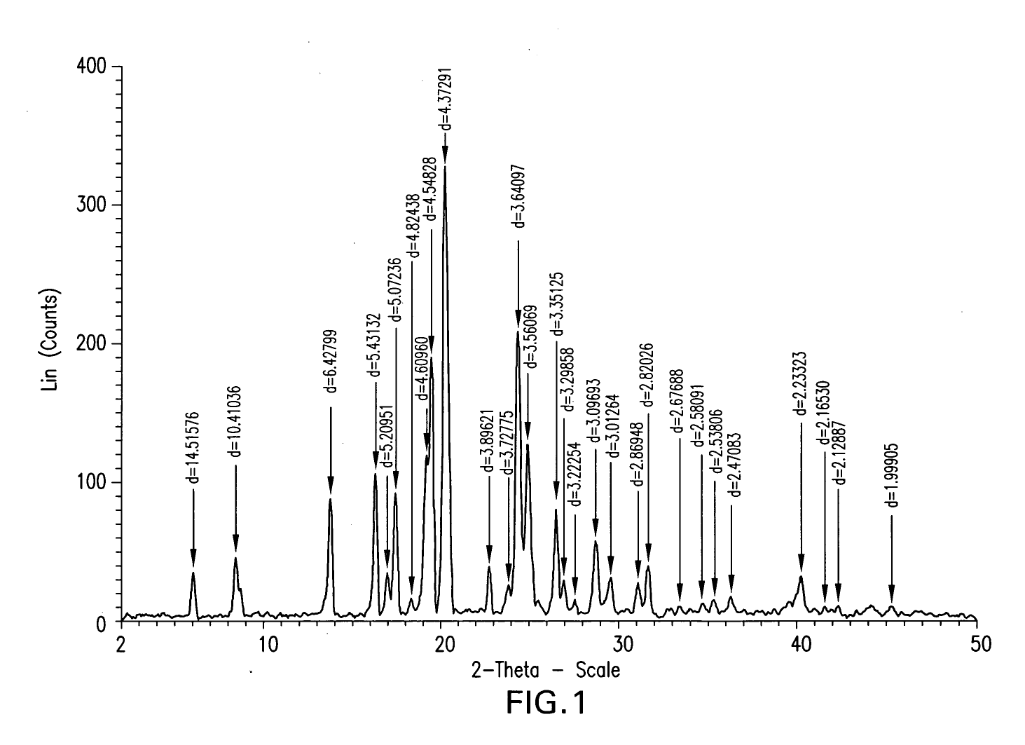

The crystalline form of compound of Formula II may also be characterized by a PXRD pattern corresponding substantially to FIG. 1 and/or by PXRD values as follows:

| Angle | | |
|---|---|---|
| (±0.2 degree 2-Theta) | d values (Angstrom) | Intensity (%) |
| 6.084 | 14.51576 | 10.4 |
| 8.487 | 10.41036 | 13.5 |
| 13.765 | 6.42799 | 26.7 |
| 16.307 | 5.43132 | 32.1 |
| 17.006 | 5.20951 | 10.2 |
| 17.470 | 5.07236 | 27.6 |
| 19.239 | 4.60960 | 36.1 |
| 19.501 | 4.54828 | 58.0 |
| 20.292 | 4.37291 | 100.0 |
| 22.806 | 3.89621 | 11.7 |
| 23.851 | 3.72775 | 7.9 |
| 24.428 | 3.64097 | 63.6 |
| 24.988 | 3.56069 | 38.4 |
| 26.577 | 3.35125 | 24.2 |
| 27.009 | 3.29858 | 8.7 |
| 28.805 | 3.09693 | 17.2 |
| 29.629 | 3.01264 | 9.4 |
| 31.144 | 2.86948 | 8.0 |
| 31.701 | 2.82026 | 11.9 |
| 36.330 | 2.47083 | 5.2 |
| 40.355 | 2.23323 | 9.7 |

One skilled in the art will appreciate that there is a small amount of variability involved in PXRD measurements. There are, in general two types of variability associated with PXRD measurements, systematic variability and random variability. Systematic variability can be caused by various factors such as: (a) variability in sample preparation (e.g., sample height), (b) instrument variability (e.g., flat sample errors), (c) calibration errors, (d) operator errors and (e) preferred orientation. When comparing different measurements, e.g., measurements made on different instruments, or even measurements made on the same instrument, but at different times, such factors often result in a shift of all the PXRD peaks in one measurement in the same direction and by the same amount relative to the other measurement. These shifts can be compensated for by applying a systematic correction factor to all the peaks of one of the measurements.

Other factors do not result in a shift of all the peaks in the same direction and by the same amount and can give rise to random variability. Random variability may be due to improperly determining the peak position. This is generally done electronically and different software may employ slightly different means of deciding precisely where a peak is located. Differences in the size of crystals, temperature differences, and differences in degree of hydration may also contribute to random variability.

The variability involved in PXRD measurements is generally smaller for small values of 2θ and larger for large values of 2θ.

Furthermore, the skilled artisan would understand that the wavelength of the radiation used when obtaining PXRD data influences the angles of diffraction observed, and therefore the positions of the PXRD peaks in a diffractogram. The skilled artisan would know how to convert peaks obtained from the use of radiation of a first wavelength to the corresponding peaks that would be obtained from the use of radiation of a second wavelength by using the Bragg equation: nλ=2d sin θ.

In practice, those skilled in the art understand that variability on the order of about ±0.2 degrees 2θ should be taken into account with respect to the values assigned to each peak. Accordingly, PXRD peak data herein are presented in the form of PXRD patterns generated using radiation of a specified wavelength and having peaks at A, B, C, etc. ±0.2 degrees 2θ. This indicates that, for the crystalline form in question, the peak at A could, in a given instrument on a given run, appear somewhere between A ±0.2 degrees 2θ, the peak at B could appear at B ±0.2 degrees 2θ, etc. Such small variability in the values assigned to individual peaks does not translate into uncertainty with respect to identifying individual crystalline forms since it is generally the particular combination of peaks within the specified ranges, as well as peaks that are unique to a particular form, and not any one particular peak, that serves to unambiguously identify crystalline forms.

The term “about” as used herein to describe the XRD peaks is intended to account for minor deviations based on variabilities discussed above.

The invention further encompasses compounds prepared according to the processes of the invention, azetidinones prepared therefrom, including ezetimibe, and pharmaceutical compositions comprising such azetidinone. The invention also encompasses a method for reducing cholesterol in a mammal comprising administering a therapeutically effective amount of such azetidinone or such pharmaceutical composition. The invention further encompasses use of a compound of the invention for the manufacture of ezetimibe, use of a process of the invention for the manufacture of ezetimibe, and use of such ezetimibe manufacture of a medicament for reducing cholesterol in a mammal.

The ezetimibe of the invention herein can be formulated into a variety of compositions for administration to humans and animals for treating diseases through the reduction of cholesterol.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of ezetimibe and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol and talc.

Carriers for use in the pharmaceutical compositions may include, but are not limited to, lactose, white sugar, sodium chloride, glucose, urea, starch, calcium carbonate, kaolin, crystalline cellulose, silicic acid, and the like.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like. Absorption accelerators may include, but are not limited to, quaternary ammonium base, sodium laurylsulfate, and the like. Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the ezetimibe forms described herein and any other solid ingredients are dissolved or suspended in a liquid carrier, such as water, vegetable oil, alcohol, polyethylene glycol, propylene glycol or glycerin. Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of ezetimibe or pharmaceutically acceptable salt thereof contained in a pharmaceutical composition for reducing cholesterol according to the present invention is not specifically restricted; however, the dose should be sufficient to treat, ameliorate, or reduce the condition. For example, ezetimibe may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of ezetimibe may be contained in an administration unit form, preferably a 10 mg tablet. Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### EXPERIMENTAL

### Crystalline Compound of Formula II

A crystalline compound of Formula II, i.e., 3-{4-[2-(4-Fluorophenyl)-5,5-dimethyl-[1,3]dioxan-2-yl]butyryl}-(4S)-phenyl-1,3-oxazolidin-2-one is determined using the following instrumentation:

Make: Simens

Model: D5000

Diffraction geometry: 2 theta

Counter: Scintillation counter

Source: Copper tube, monochromatic graphite crystal.

The crystalline form of the compound of Formula II has the following PXRD values.

| Angle | | |
|---|---|---|
| (±0.2 degree 2-Theta) | d values (Angstrom) | Intensity (%) |
| 6.084 | 14.51576 | 10.4 |
| 8.487 | 10.41036 | 13.5 |
| 13.765 | 6.42799 | 26.7 |
| 16.307 | 5.43132 | 32.1 |
| 17.006 | 5.20951 | 10.2 |
| 17.470 | 5.07236 | 27.6 |
| 19.239 | 4.60960 | 36.1 |
| 19.501 | 4.54828 | 58.0 |
| 20.292 | 4.37291 | 100.0 |
| 22.806 | 3.89621 | 11.7 |
| 23.851 | 3.72775 | 7.9 |
| 24.428 | 3.64097 | 63.6 |
| 24.988 | 3.56069 | 38.4 |
| 26.577 | 3.35125 | 24.2 |
| 27.009 | 3.29858 | 8.7 |
| 28.805 | 3.09693 | 17.2 |
| 29.629 | 3.01264 | 9.4 |
| 31.144 | 2.86948 | 8.0 |
| 31.701 | 2.82026 | 11.9 |
| 36.330 | 2.47083 | 5.2 |
| 40.355 | 2.23323 | 9.7 |

### Synthesis of Intermediate-XII (3-{4-[2-(4-fluorophenyl)-5,5-dimethyl-[1,3]dioxan-2-yl]butyryl}(4S)-phenyl-1,3oxazolidin-2-one)

### Example-1a

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), toluene (1.0 L), neopentyl glycol (0.264 kg, 2.53 mol) and pyridinium hydrobromide (0.0045 kg, 0.028 mol) at 20° C. to 25° C. The resulting mixture was refluxed for 5 hrs at 110-115° C. The reaction was monitored by TLC/HPLC. After completion of reaction, the mixture was cooled to ambient temperature and washed with water (0.5 L) twice. The organic layer was concentrated to an oil, which was crystallized from isopropyl alcohol to afford 0.097 kg of 3-{4-[2-(4-fluorophenyl)-5,5-dimethyl-[1,3]dioxan-2-yl]butyryl}(4S)-phenyl oxazolidin-2-one.

### Example-1b

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), toluene (1.0 L), neopentyl glycol (0.264 kg, 2.53 mol) and pyridinium para toluenesulfonate (0.0035 kg, 0.014 mol) at 20-25° C. The resulting mixture was refluxed for 5 hrs at 110-115° C. The reaction was monitored by TLC/HPLC. After completion of reaction, the mixture was cooled to ambient temperature and washed with water (0.5 L) twice. The organic layer was concentrated to an oil, which was crystallized from ethanol to afford 0.095 kg of 3-{4-[2-(4-fluorophenyl)-5,5-dimethyl-[1,3]dioxan-2-yl]butyryl}(4S)-phenyl oxazolidin-2-one.

### Example-1c

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), toluene (1.0 L), neopentyl glycol (0.264 kg, 2.53 mol) and para toluene sulfonic acid (0.0025 kg, 0.014 mol) at 20-25° C. The resulting mixture was refluxed for 5 hrs at 110-115° C. The reaction was monitored by TLC/HPLC. After completion of the reaction, the mixture was cooled to ambient temperature and washed with water (0.5 L) twice. The organic layer was concentrated to an oil, which was crystallized from isopropyl alcohol to obtain 0.097 kg of 3-{4-[2-(4-fluorophenyl)-5,5-dimethyl-[1,3]dioxan-2-yl]butyryl}(4S)-phenyl oxazolidin-2-one.

### Example-1d

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), MDC (1.0 L), neopentyl glycol (0.264 kg, 2.53 mol) and trimethyl silyl chloride (0.03 kg, 0.28 mol) at 20-25° C. The resulting mixture was refluxed for 5 hrs at 110-115° C. The reaction was monitored by TLC/HPLC. After completion of the reaction, the mixture was cooled to ambient temperature and washed with water (0.5 L) twice. The organic layer was concentrated to an oil, which was crystallized from isopropyl alcohol to obtain 0.092 kg of 3-{4-[2-(4-fluorophenyl)-5,5-dimethyl-[1,3]dioxan-2-yl]butyryl}(4S)-phenyl oxazolidin-2-one.

### Example-1e

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), MDC (1.0 L), neopentyl glycol (0.264 kg, 2.53 mol) and BF₃-etherate (0.088 kg, 0.62 mol) at 20-25° C. The resulting mixture was refluxed for 5 hrs at 110-115° C. The reaction was monitored by TLC/HPLC. After completion of the reaction, the mixture was cooled to ambient temperature and washed with water (0.5 lt) twice. The organic layer was concentrated to an oil, which was crystallized from isopropyl alcohol to obtain 0.090 kg of 3-{4-[2-(4-fluorophenyl)-5,5-dimethyl-[1,3]dioxan-2-yl]butyryl}(4S)-phenyl oxazolidin-2-one

### Example-1f

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), cyclohexane (0.5 L), neopentyl glycol (0.264 kg, 2.53 mol) and pyridinium hydrobromide (0.0045 kg, 0.028 mol) at 20° C. to 25° C. The resulting mixture was refluxed for 5 hrs at 80-85° C. The reaction was monitored by TLC/HPLC. After completion of reaction, the mixture was cooled and washed with water (0.5 L) twice at 50-70° C. The organic layer was concentrated to an oil, which was crystallized from isopropyl alcohol to afford 0.104 kg of 3-{4-[2-(4-fluorophenyl)-5,5-dimethyl-[1,3]dioxan-2-yl]butyryl}(4S)-phenyl oxazolidin-2-one.

### Example-1g

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), cyclohexane (0.5 L), neopentyl glycol (0.264 kg, 2.53 mol) and pyridinium para toluenesulfonate (0.0035 kg, 0.014 mol) at 20-25° C. The resulting mixture was refluxed for 5 hrs at 80-85° C. The reaction was monitored by TLC/HPLC. After completion of reaction, the mixture was cooled and washed with water (0.5 L) twice at 50-70° C. The organic layer was concentrated to an oil, which was crystallized from ethanol to afford 0.098 kg of 3-{4-[2-(4-fluorophenyl)-5,5-dimethyl-[1,3]dioxan-2-yl]butyryl}(4S)-phenyl oxazolidin-2-one.

### Example-1 h

A 4-necked round-bottomed flask fitted with a thermometer pocket and N₂ inlet was charged with 3-[5-(4-fluorophenyl)-1,5-dioxopentyl]-4-phenyl-(4S)-2-oxazolidinone (0.1 kg, 0.281 mol), cyclohexane (0.5 L), neopentyl glycol (0.264 kg, 2.53 mol) and para toluene sulfonic acid (0.0025 kg, 0.014 mol) at 20-25° C. The resulting mixture was refluxed for 5 hrs at 80-85° C. The reaction was monitored by TLC/HPLC. After completion of the reaction, the mixture was cooled and washed with water (0.5 L) twice at 50-70° C. The organic layer was concentrated to an oil, which was crystallized from isopropyl alcohol to obtain 0.097 kg of 3-{4-[2-(4-fluorophenyl)-5,5-dimethyl-[1,3]dioxan-2-yl]butyryl}(4S)-phenyl oxazolidin-2-one.

### Example-1g

To a 4-necked, 3-liter, round-bottomed flask fitted with a thermometer pocket and N₂ inlet was added 4-[2-(4-fluorophenyl)-5,5-dimethyl-[1,3]-dioxan-2-yl]-butyric acid (0.1 kg, 0.38 mol), MDC (0.5 L), TEA (0.061 kg, 0.61 mol) and pivaloyl chloride (0.49 kg, 0.41 mol) at 20-25° C. The resulting mixture was stirred for 2 hrs at 20 to 25° C. and monitored by TLC/HPLC. After the completion of the reaction, (S)-4-phenyl-2-oxazolidinone (0.06 kg, 0.37 mol), DMAP (0.008 kg, 0.07 mol) and DMF (0.05 L) were added to the mixture at 20 to 25° C. and refluxed for 7 hrs. The reaction was monitored by TLC/HPLC. After completion of the reaction, the mixture was cooled to 20 to 25° C. and 2N sulfuric acid (0.4 L) was added to the mixture at 20 to 25° C. The organic layer was separated and washed with 10% aqueous sodium bicarbonate solution (0.5 L), DM water (2×0.5 L) and brine solution (0.5 L). The organic layer was then concentrated to an oil, which was crystallized from isopropyl alcohol to afford 0.13 kg of 3-{4-[2-(4-fluorophenyl)-5,5-dimethyl-[1,3]dioxan-2-yl]butyryl}(4S)-phenyl oxazolidin-2-one.

### Synthesis of Intermediate-X (3-{(2R,3S)-3-[(4-fluorophenyl)amino]-2-[2-(4-fluorophenyl)-5,5-dimethyl-1,3-dioxan-2-yl]-3-[4-(benzyloxy)phenyl]propanoyl}-(4S)-4-phenyl-1,3-oxazolidin-2-one)

### Example-2a

To a 4-necked, 5-liter round-bottomed flask fitted with a thermometer pocket and N₂ gas inlet was added 3-{4-[2-(4-fluorophenyl)-5,5-dimethyl-[1,3]dioxan-2-yl]butyryl}(4S)-phenyl oxazolidin-2-one (0.1 kg, 0.226 mol), MDC (1.5 L). The resulting mixture was cooled to −25 to −30° C. DIPEA (0.058 kg, 0.44 mol), benzyl imine ((4-benzyloxybenzylidene)fluoroaniline) (0.103 kg, 0.34 mol), titanium tetrachloride (0.047 kg, 0.24 mol) at −25 to −30° C. were then added to the reaction mixture. The reaction mixture was then maintained at −25 to −30° C. for 3 hrs. and monitored by TLC/HPLC. After completion of the reaction, glacial acetic acid (0.1 L) was added slowly to the reaction mixture while maintaining the reaction temperature between −25 and −30° C. The temperature was raised to 0-5° C. and 7% aqueous tartaric acid solution (1.5 L) was added at this temperature. The mixture was stirred for 1 hr. at 0-5° C. and then allowed to warm gradually to room temperature. 20% aqueous sodium bisulfite solution (0.5 L) was then added to the mixture and the mixture was maintained with stirring for 30 min. The organic layer was separated and washed with water (500 ml) and 10% aqueous sodium bicarbonate solution (2×500 ml) in succession. The organic solvent was distilled from the organic layer and the resulting residue was crystallized from absolute ethanol to afford 0.083 kg of 3-{(2R,3S)-3-[(4-fluorophenyl)amino]-2-[2-(4-fluorophenyl)-5,5-dimethyl-1,3-dioxan-2-yl]-3-[4-(benzyloxy)phenyl]propanoyl}-(4S)-4-phenyl-1,3-oxazolidin-2-one.

### Example-2b

To a 4-necked round-bottomed flask fitted with a thermometer pocket and N₂ gas inlet was added 3-{4-[2-(4-fluorophenyl)-5,5-dimethyl-[1,3]dioxan-2-yl]butyryl}(4S)-phenyl oxazolidin-2-one (0.1 kg, 0.226 mol), MDC (1.5 L), and hydroxy imine (0.097 kg, 0.45 mol). The resulting mixture was then cooled to −5 to −10° C. DIPEA (0.058 kg, 0.44 mol) and trimethyl silyl chloride (0.037 kg, 0.34 mol) were then added to the mixture and the mixture was stirred for 0 hr at −5 to −10° C. The reaction mixture was then cooled to −20 to −25° C. and titanium tetrachloride (0.047 kg, 0.24 mol) was added. The reaction mixture was maintained at −25 to −30° C. for 3 hrs. and monitored by TLC/HPLC. After completion of the reaction, glacial acetic acid (0.1 L) was added slowly to the reaction mixture while maintaining the reaction temperature between −25 and −30° C. The temperature was then raised to 0-5° C. and 7% aqueous tartaric acid solution (1.5 L) was added at this temperature. The mixture was stirred for 1 hr. at 0-5° C. than allowed to warm gradually to room temperature. 20% aqueous sodium bisulfite solution (0.5 L) was then added to the mixture and the mixture was maintained with stirring for 30 min. The organic layer was separated and washed with water (500 ml) and 10% aqueous sodium bicarbonate solution (2×500 ml) in succession. The organic solvent was distilled from the organic layer and the residue was crystallized in absolute ethanol to afford 0.070 kg 3-{(2R,3S)-3-[(4-fluorophenyl)amino]-2-[2-(4-fluorophenyl)-5,5-dimethyl-1,3-dioxan-2-yl]-3-[4-(trimethylsilanyloxy)phenyl]propanoyl}-(4S)-4-phenyl-1,3-oxazolidin-2-one.

### Example-2c

To a 4-necked, 5-liter, round-bottomed flask fitted with thermometer pocket and N₂ gas inlet was added 3-{4-[2-(4-fluorophenyl)-5,5-dimethyl-[1,3]dioxan-2-yl]butyryl}(4S)-phenyl oxazolidin-2-one (0.1 kg, 0.226 mol), and MDC (1.5 L). The resulting mixture was cooled to −25 to −30° C. DIPEA (0.058 kg, 0.44 mol), benzyl imine ((4-benzyloxybenzylidene)fluoroaniline) (0.103 kg, 0.34 mol), titanium tetrachloride (0.047 kg, 0.24 mol) and titanium tetraisopropoxide (0.0206 kg, 0.0724 mol) were then added to the mixture at −25 to −30° C. The reaction mixture was maintained at −25 to −30° C. for 3 hrs. and monitored by TLC/HPLC. After completion of the reaction, glacial acetic acid (0.1 L) was added slowly to the reaction mixture while maintaining the reaction temperature between −25 and −30° C. The temperature was raised to 0-5° C. and 7% aqueous tartaric acid solution (1.5 L) was added at this temperature. The mixture was stirred for 1 hr. at 0-5° C. and then allowed to warm gradually to room temperature. 20% aqueous sodium bisulfite solution (0.5 L) was added to the mixture and the mixture was maintained with stirring for 30 min. The organic layer was separated and washed with water (500 ml) and 10% aqueous sodium bicarbonate solution (2×500 ml) in succession. The organic solvent was distilled from the organic layer and the residue was crystallized in absolute ethanol to afford 0.085 kg 3-{(2R,3S)-3-[(4-fluorophenyl)amino]-2-[2-(4-fluorophenyl)-5,5-dimethyl-1,3-dioxan-2-yl]-3-[4(benzyloxy)phenyl]propanoyl}-(4S)-4-phenyl-1,3-oxazolidin-2-one.

### Synthesis of Intermediate-IV (4(S)-(4-Benzyloxyphenyl)-1-(4-fluorophenyl)-3(R)-{2-[2-(4-fluorophenyl)-5,5-dimethyl-[1,3]-dioxan-2-yl]-ethyl}azetidine-2-one)

### Example-3a

To a 4-necked, 2-liter, round-bottomed flask fitted with a thermometer pocket and N₂ inlet were added 3-{(2R,3S)-3-[(4-fluorophenyl)amino]-2-[2-(4-fluorophenyl)-5,5-dimethyl-1,3-dioxan-2-yl]-3-[4-(benzyloxy)phenyl]propanoyl}-(4S)-4-phenyl-1,3-oxazolidin-2-one (0.1 kg, 0.134 mol), THF (1.0 L), and N,O-Bistrimethylsilyl acetamide (0.068 kg, 0.33 mol) at 20 to 25° C. The resulting mixture was refluxed for 4 hrs. Then tetrabutylammoniumfluoride (3.5 g, 0.013 mol) was added to the mixture at reflux and maintained for 1 hr. The reaction was monitored by TLC/HPLC. After completion of the reaction, the mixture was cooled to 20-25° C. and glacial acetic acid (0.005 L) was added. The reaction mixture was then concentrated and crystallized from isopropyl alcohol (1.0 L) to afford 0.065 kg. of 4(S)-(4-Benzyloxyphenyl)-1-(4-fluorophenyl)-3(R)-{2-[2-(4-fluorophenyl)-5,5-dimethyl-[1,3]-dioxan-2-yl]-ethyl}azetidine-2-one.

### Example-3b

To a 4-necked round-bottomed flask fitted with a thermometer pocket and N₂ inlet were added 3-{(2R,3S)-3-[(4-fluorophenyl)amino]-2-[2-(4-fluorophenyl)-5,5-dimethyl-1,3-dioxan-2-yl]-3-[4-(trimethylsilanyloxy)phenyl]propanoyl}-(4S)-4-phenyl-1,3-oxazolidin-2-one (0.1 Kg, 0.135 mol), THF (1.0 L), N,O-Bistrimethylsilyl acetamide (0.068 kg, 0.33 mol) at 20 to 25° C. and refluxed for 4 hrs at 65 to 70° C. The resulting mixture was then cooled to 60-65° C. and tetrabutylammoniumfluoride (3.6 gm, 0.013 mol) was added. The mixture was then heated back to reflux and maintained at reflux for 1 hr. The reaction was monitored by TLC/HPLC. After completion of the reaction, the mixture was cooled to 20-25° C. and concentrated to oil. The obtained oil was dissolved in methylene dichloride (0.5 L) and washed with DM water (2×0.5 L) and brine solution (0.5 L) in succession. The organic layer was then concentrated to afford a solid, which was crystallized from absolute ethanol (1.0 L) to yield 0.06 kg of 4(S)-(4-trimethylsilanyloxyl)-1-(4-fluorophenyl)-3(R)-{2-[2-(4-fluorophenyl)-5,5-dimethyl-[1,3]-dioxan-2-yl]-ethyl}azetidine-2-one.

### Example-3c

To a 4-necked (2 L) round-bottomed flask fitted with thermometer pocket and N₂ inlet were added 3-{(2R,3S)-3-[(4-fluorophenyl)amino]-2-[2-(4-fluorophenyl)-5,5-dimethyl-1,3-dioxan-2-yl]-3-[4-(benzyloxy)phenyl]propanoyl}-(4S)-4-phenyl-1,3-oxazolidin-2one (0.1 Kg, 0.134 mol), THF (1.0 L), N,O-Bistrimethylsilyl acetamide (0.068 kg, 0.33 mol) at 20 to 25° C. The content was refluxed for 4 hrs and cooled to 25 to 30° C. Tetrabutylammoniumfluoride (3.5 gm, 0.013 mol) was added, and the reaction mixture was maintained for 1 hr. The reaction was monitored by TLC/HPLC. After completion of reaction glacial acetic acid (0.005 L) was added, and the reaction mixture was concentrated and crystallized in isopropyl alcohol (1.0 L) to produce 0.07 kg of 4(S)-(4-Benzyloxyphenyl)-1-(4-fluorophenyl)-3(R)-{2-[2-(4-fluorophenyl)-5,5-dimethyl-[1,3]-dioxan-2-yl]-ethyl}azetidine-2-one.

### Example-3d

To a 4-necked round-bottomed flask fitted with a thermometer pocket and N₂ inlet was added 3-{(2R,3S)-3-[(4-fluorophenyl)amino]-2-[2-(4-fluorophenyl)-5,5-dimethyl-1,3-dioxan-2-yl]-3-[4-(trimethylsilanyloxy)phenyl]propanoyl}-(4S)-4-phenyl-1,3-oxazolidin-2-one (0.1 kg, 0.135 mol) in toluene (0.4 L). To this mixture was added N,O-bistrimethylsilyl acetamide (0.055 kg, 0.27 mol) and tetra butyl ammonium fluoride (1M solution in THF) (0.013 L, 0.051 mol) at 45 to 50° C., and maintained at 45 to 50° C. for 2 hr. The reaction was monitored by TLC/HPLC. After completion of the reaction, added 1N aq. HCl (0.04 L) solution at 40-50° C. Organic layer was separated and washed with 8% sodium bicarbonate solution (0.4 L) and water (0.4 L) at 40-50° C. The organic layer was then concentrated to afford a solid, which was crystallized from methanol (0.6 L) to yield 0.057 kg of 4(S)-(4-trimethylsilanyloxyl)-1-(4-fluorophenyl)-3(R)-{2-[2-(4-fluorophenyl)-5,5-dimethyl-[1,3]-dioxan-2-yl]-ethyl}azetidine-2-one. Distilled out mother liquor to an oil and which was crystallized in toluene (0.2 L) to yield 0.01 kg of (S)-4-phenyl-2-oxazolidinone.

### Synthesis of Intermediate-II (4(S)-(4-Benzyloxyphenyl)-1-(4-fluorophenyl)-3(R)-[3-(4-fluorophenyl)-3-oxopropyl]-azetidine-2-one)

### Example-4a

To a 4-necked, 2-liter, round-bottomed flask fitted with a thermometer pocket and N₂ inlet was added 4(S)-(4-Benzyloxyphenyl)-1-(4-fluorophenyl)-3(R)-{2-[2-(4-fluorophenyl)-5,5-dimethyl-1,3-dioxan-2-yl]-ethyl}azetidine-2-one (0.1 kg, 0.17 mol) in formic acid (1.5 L) at 20 to 25° C. The resulting mixture was stirred for 1 hr. and monitored by TLC/HPLC. After completion of the reaction, the mixture was concentrated to oil. The oil was dissolved in MDC (0.5 L) and washed with DM water (0.5 L) twice. The MDC was then distilled to afford 0.072 kg. of 4(S)-(4-Benzyloxyphenyl)-1-(4-fluorophenyl)-3(R)-[3-(4-fluorophenyl)-3-oxopropyl]-azetidine-2-one.

### Example-4b

To a 4-necked, 2-liter, round-bottomed flask fitted with a thermometer pocket and N₂ inlet was added 4-[2-(4-fluorophenyl)-5,5-dimethyl-[1,3]-dioxan-2-yl]-butyric acid (0.1 kg, 0.17 mol) in formic acid (1.5 L) at 20 to 25° C. The resulting mixture was stirred for 1 hr. and monitored by TLC/HPLC. After completion of the reaction, the mixture was concentrated to an oil. The oil was dissolved in MDC (0.5 L) and washed with DM water (0.5 L) twice. The MDC was distilled to afford an oil which was crystallized from IPA to afford 0.07 kg. of 4(S)-(4-Benzyloxyphenyl)-1-(4-fluorophenyl)-3(R)-[3-(4-fluorophenyl)-3-oxopropyl]-azetidine-2-one.

### Example-4c

To a 4-necked, 2-liter, round-bottomed flask fitted with a thermometer pocket and N₂ inlet was added 4-[2-(4-fluorophenyl)-5,5-dimethyl-[1,3]-dioxan-2-yl]-butyric acid (0.1 kg, 0.17 mol) in methylene chloride (0.15 L) and formic acid (0.2 L) at 25 to 30° C. The resulting mixture was stirred for 4 hr and monitored by TLC/HPLC. After completion of the reaction, the mixture was washed with DM water (0.5 L) twice. The methylene chloride was distilled to afford oil which was crystallized from ethanol (0.3 L) to afford 0.077 kg of 4(S)-(4-benzyloxyphenyl)-1-(4-fluorophenyl)-3(R)-[3-(4-fluorophenyl)-3-oxopropyl]-azetidine-2-one.

### Synthesis of Intermediate-XIV (4(S)-(4-Benzyloxyphenyl)-1-(4-fluorophenyl)-3(R)-[3-(4-fluorophenyl)-3(S)-hydroxypropyl]-azetidine-2-one)

### Example-5a

To a 4-necked, 5-liter, round-bottomed flask fitted with a thermometer pocket and N₂ inlet were added borane dimethylsulfide complex (0.0167 kg, 0.22 mol), (R)-2-Methyl-CBS-oxazaborolidine (0.0122 kg, 0.044 mol), methanesulfonic acid (0.38 g, 0.004 mol) and THF (0.4 L) at 5 to 10° C. The resulting mixture was cooled to −15° C. and a solution of 4(S)-(4-Benzyloxyphenyl)-1-(4-fluorophenyl)-3(R)-[3-(4-fluorophenyl)-3-oxopropyl]-azetidine-2-one (0.1 Kg, 0.020 mol) in THF (0.4 L) was added over a period of 45 to 60 minutes, while the mixture was gradually being warmed to a temperature of 0° C. The mixture was stirred for 30 min at −5 to 0° C. and monitored by TLC/HPLC. After completion of the reaction, methanol (0.1 L) followed by 1N HCl (400 ml) were added at 0° C. and the mixture was stirred for 30 min. The reaction mixture was then extracted with ethyl acetate (2×500 ml) and washed with DM Water (0.5 L). The organic layer was concentrated to an oil, which was crystallized from ethyl alcohol to afford 0.05 kg of 4(S)-(4-Benzyloxyphenyl)-1-(4-fluorophenyl)-3(R)-[3-(4-fluorophenyl)-3(S)-hydroxypropyl]-azetidine-2-one.

### Example-5b

To a 4-necked, 5-liter round-bottomed flask fitted with a thermometer pocket and N₂ inlet were added borane dimethylsulfide complex (0.016 kg, 0.22 mol), (R)-2-methyl-CBS-oxazaborolidine (1M in toluene, 0.044 L, 0.044 mol), and methanesulfonic acid (0.38 g, 0.004 mol) in THF (0.4 L) at 0 to 5° C. A solution of 4(S)-(4-Benzyloxyphenyl)-1-(4-fluorophenyl)-3(R)-[3-(4-fluorophenyl)-3-oxopropyl]-azetidine-2-one (0.1 kg, 0.20 mol) in THF (0.4 L) was then added to the mixture over a period of 45 to 60 min at 0-5° C. The mixture was then stirred for 30-45 min at 0-5° C. and the reaction was monitored by TLC/HPLC. After completion of the reaction, methanol (0.1 L) followed by 1N HCl (400 ml) were added at 0° C. and the mixture was stirred for 30 min. The mixture was then extracted with ethyl acetate (2×500 ml) and washed with DM Water (0.5 L). The organic layer was concentrated to oil, which was crystallized from ethyl alcohol to afford 0.05 kg of 4(S)-(4-Benzyloxyphenyl)-1-(4-fluorophenyl)-3(R)-[3-(4-fluorophenyl)-3(S)-hydroxypropyl]-azetidine-2-one.

### Example-5c

To a solution of 4(S)-(4-Benzyloxyphenyl)-1-(4-fluorophenyl)-3(R)-[3-(4-fluorophenyl)-3-oxopropyl]-azetidine-2-one (0.1 kg, 0.20 mol) in THF (0.8 L) were added (R)-2-Methyl-CBS-oxazaborolidine (1M in toluene, 0.044 L, 0.044 mol), and methanesulfonic acid (0.38 g, 0.004 mol) at 20-25° C. The resulting mixture was then cooled to −20 to −25° C. and borane dimethylsulfide complex (0.016 kg, 0.22 mol) was added to the mixture over a period of 30-45 minutes at −20 to −25° C. The mixture was maintained for 2 hrs at −20 to −25° C. and monitored by TLC/HPLC. After completion of the reaction, methanol (0.1 L) followed by 1N HCl (400 ml) were added to the mixture at 0° C., and the mixture was stirred for 30 min. The mixture was then extracted with ethyl acetate (2×500 ml) and washed with DM Water (0.5 L). The organic layer was concentrated to an oil, which was crystallized from a mixture of n-heptane and ethyl alcohol to yield 0.05 kg of 4(S)-(4-Benzyloxyphenyl)-1-(4-fluorophenyl)-3(R)-[3-(4-fluorophenyl)-3(S)-hydroxypropyl]-azetidine-2-one.

### Example-5d

To a solution of 4(S)-(4-hydroxyphenyl)-1-(4-fluorophenyl)-3(R)-[3-(4-fluorophenyl)-3oxopropyl]azetidine-2-one (0.1 kg, 0.24 mol) in THF (0.8 L) was added trimethyl silyl chloride (0.032 kg, 0.29 mol) at −5 to −10° C. The resulting mixture was then stirred for 1 hour. After completion of silylation, (R)-2-Methyl-CBS-oxazaborolidine (1 M in toluene, 0.015 kg, 0.054 mol) and methanesulfonic acid (0.47 g, 0.005 mol) were added to the mixture at 20-25° C. The mixture was then cooled to −20 to −25° C. Borane dimethylsulfide complex (0.0204 kg, 0.27 mol) was then added to the mixture over a period of 30-45 min at −20 to −25° C. The mixture was maintained for 2 hrs at −20 to −25° C. and monitored by TLC/HPLC. After completion of the reaction, methanol (0.1 L) followed by 1N HCl (400 ml) were added to the mixture at 0° C., and the mixture was stirred for 30 minutes. The mixture was then extracted with ethyl acetate (2×500 ml) and washed with DM Water (0.5 L). The organic layer was concentrated to oil, which was crystallized from ethyl alcohol to yield 0.048 kg of 1-(4-fluorophenyl)-3(R)-[3-(4-fluorophenyl)-3(S)-hydroxypropyl]-4(S)-hydroxyphenyl-azetidine-2-one, i.e., ezetimibe.

### Example-5e

To a 4-necked, 5-liter, round-bottomed flask fitted with a thermometer pocket and N₂ inlet were added 4(S)-(4-Benzyloxyphenyl)-1-(4-fluorophenyl)-3(R)-[3-(4-fluorophenyl)-3-oxopropyl]-azetidine-2-one (0.1 kg, 0.20 mol), THF (0.8 L) and (R)-2-Methyl-CBS-oxazaborolidine (1 M in toluene, 0.044 L, 0.044 mol) at 20-25° C. The resulting mixture was then cooled to −20 to −25° C. and borane dimethylsulfide complex (0.016 kg, 0.22 mol) was added to the mixture drop-wise over a period of 0.5 hrs. The mixture was then maintained for 2 hrs at −20 to −25° C. and monitored by TLC/HPLC. After completion of the reaction, methanol (0.1 L) followed by 1N HCl (400 ml) were added at 0° C. The mixture was then stirred for 30 minutes and extracted with ethyl acetate (2×500 ml) and washed with DM water (0.5 L). The organic layer was concentrated to oil, which was crystallized from a mixture of n-heptane and ethyl alcohol to yield 0.05 kg of 4(S)-(4-Benzyloxyphenyl)-1-(4-fluorophenyl)-3(R)-[3-(4-fluorophenyl)-3(S)-hydroxypropyl]-azetidine-2-one.

### Example-5f

To a 4-necked, 5-liter, round-bottomed flask fitted with a thermometer pocket and N₂ inlet were added MDC (0.2 L) and borane dimethylsulfide complex (0.0167 kg, 0.22 mol). The resulting mixture was then cooled to −5° C. to 0° C. (R)-2-Methyl-CBS-oxazaborolidine (1M in toluene, 0.044 L, 0.044 mol) was added to the mixture and the mixture was stirred for 15 min at 0° C. A solution of 4(S)-(4-Benzyloxyphenyl)-1-(4-fluorophenyl)-3(R)-[3-(4-fluorophenyl)-3-oxopropyl]-azetidine-2-one (0.1 kg, 0.020 mol) in MDC (0.3 L) was then slowly added to the mixture over a period of 3-4 hrs. The reaction temperature was then maintained between −5° C. and 0° C. for 1 to 2 hrs, and the reaction was monitored by TLC/HPLC. After completion of the reaction, methanol (0.1 L), followed by 1N HCl (400 ml) were added to the mixture at 0° C., and the mixture was stirred for 30 minutes. The mixture was then extracted with ethyl acetate (2×500 ml) and washed with DM water (0.5 L). The organic layer was concentrated to oil, which was crystallized from ethyl alcohol to afford 0.05 kg of 4(S)-(4-Benzyloxyphenyl)-1-(4-fluorophenyl)-3(R)-[3-(4-fluorophenyl)-3(S)-hydroxypropyl]-azetidine-2-one.

### Synthesis of Intermediate-VI (4-[2-(4-fluorophenyl)-5,5-dimethyl-[1,3]-dioxan-2-yl]-butyric acid-3-hydroxy-2,2-dimethylpropyl ester)

### Example-6a

To a 4-necked, round-bottomed flask fitted with a thermometer pocket were added 4-(4-fluorophenyl)-4-oxo-butyric acid (0.1 kg, 0.47 mol), toluene (1.5 L), neopentyl glycol (0.496 kg, 4.72 mol) and p-toluene sulfonic acid (0.009 kg, 0.047 mol) at 20-25° C. The resulting mixture was refluxed for 3 hours at 110-115° C. The reaction was monitored by TLC/HPLC. After completion of the reaction, the mixture was cooled to 20-25° C. and washed with DM Water (3×1.5 L). The organic layer was concentrated to an oil to yield 0.18 kg of 4-[2-(4-fluorophenyl)-5,5-dimethyl-[1,3]-dioxan-2-yl]-butyric acid-3-hydroxy-2,2-dimethylpropyl ester.

### Example-6b

To a 4-necked round-bottomed flask fitted with a thermometer pocket were added 4-(4-fluorophenyl)-4-oxo-butyric acid (0.1 kg, 0.47 mol), toluene (1.5 L), neopentyl glycol (0.496 kg, 4.72 mol) and pyridinium hydrobromide (0.0076 kg, 0.047 mol) at 20-25° C. The resulting mixture was then refluxed for 3 hours at 110-115° C. The reaction was monitored by TLC/HPLC. After completion of the reaction, the mixture was cooled to 20-25° C. and washed with DM Water (3×1.5 L). The organic layer was concentrated to an oil to yield 0.16 kg of 4-[2-(4-fluorophenyl)-5,5-dimethyl-[1,3]-dioxan-2-yl]-butyric acid-3-hydroxy-2,2-dimethylpropyl ester.

### Example 6c

To a 4-necked round-bottomed flask fitted with a thermometer pocket were added 4-(4-fluorophenyl)-4-oxo-butyric acid (0.1 kg, 0.47 mol), toluene (1.5 L), neopentyl glycol (0.496 kg, 4.72 mol) and pyridinium p-toluene sulfonate (0.012 kg, 0.047 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was concentrated to an oil to yield 0.17 kg of 4-[2-(4-fluorophenyl)-5,5-dimethyl-[1,3]-dioxan-2-yl]-butyric acid-3-hydroxy-2,2-dimethylpropyl ester.

### Example-6d

To a 4-necked round-bottomed flask fitted with a thermometer pocket were added methyl-4-(4-fluorophenyl)-4-oxo-butyrate (0.1 kg, 0.44 mol), toluene (1.5 L), neopentyl glycol (0.465 kg, 4.46 mol) and p-toluene sulfonic acid (0.009 kg, 0.047 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.12 kg of 4-[2-(4-fluorophenyl)-5,5-dimethyl-[1,3]-dioxan-2-yl]-butyric acid methyl ester.

### Example-6e

To a 4-necked round-bottomed flask fitted with a thermometer pocket were added methyl-4-(4-fluorophenyl)-4-oxo-butyrate (0.1 kg, 0.44 mol), toluene (1.5 L), neopentyl glycol (0.465 kg, 4.46 mol) and pyridinium hydrobromide (0.0071 kg, 0.044 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.11 kg of 4-[2-(4-fluorophenyl)-5,5-dimethyl-[1,3]-dioxan-2-yl]-butyric acid methyl ester.

### Example-6f

To a 4-necked, round-bottomed flask fitted with a thermometer pocket were added methyl-4-(4-fluorophenyl)-4-oxo-butyrate (0.1 kg, 0.44 mol), toluene (1.5 L), Neopentyl glycol (0.465 kg, 4.46 mol) and pyridinium p-toluene sulfonate (0.011 kg, 0.043 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.11 kg of 4-[2-(4-fluorophenyl)-5,5-dimethyl-[1,3]-dioxan-2-yl]-butyric acid methyl ester.

### Example-6g

To a 4-necked round-bottomed flask fitted with a thermometer pocket were added ethyl-4-(4-fluorophenyl)-4-oxo-butyrate (0.1 kg, 0.42 mol), toluene (1.5 L), neopentyl glycol (0.437 kg, 4.20 mol) and p-toluene sulfonic acid (0.008 kg, 0.042 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.12 kg of 4-[2-(4-fluorophenyl)-5,5-dimethyl-[1,3]-dioxan-2-yl]-butyric acid ethyl ester.

### Example-6h

To a 4-necked round-bottomed flask fitted with a thermometer pocket were added ethyl-4-(4-fluorophenyl)-4-oxo-butyrate (0.1 kg, 0.42 mol), toluene (1.5 L), neopentyl glycol (0.465 kg, 4.46 mol) and pyridinium hydrobromide (0.0067 kg, 0.042 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.11 kg of 4-[2-(4-fluorophenyl)-5,5-dimethyl-[1,3]-dioxan-2-yl]-butyric acid ethyl ester.

### Example-6i

To a 4-necked round-bottomed flask fitted with a thermometer pocket were added ethyl-4-(4-fluorophenyl)-4-oxo-butyrate (0.1 kg, 0.44 mol), toluene (1.5 L), neopentyl glycol (0.465 kg, 4.46 mol) and pyridinium p-toluene sulfonate (0.011 kg, 0.042 mol) at 20-25° C. The resulting mixture was then refluxed for 3 hours at 110-115° C. The reaction was monitored by TLC/HPLC. After completion of the reaction, the mixture was cooled to 20-25° C. and washed with DM water (3×1.5 L). The organic layer was then concentrated to an oil to yield 0.10 kg of 4-[2-(4-fluorophenyl)-5,5-dimethyl-[1,3]-dioxan-2-yl]-butyric acid ethyl ester.

### Example-6j

To a 4-necked, round-bottomed flask fitted with a thermometer pocket were added 4-(4-fluorophenyl)-4-oxo-butyric acid (0.1 kg, 0.47 mol), cyclohexane (0.5 L), neopentyl glycol (0.496 kg, 4.72 mol) and p-toluene sulfonic acid (0.009 kg, 0.047 mol) at 20-25° C. The resulting mixture was refluxed for 3 hours at 80-85° C. The reaction was monitored by TLC/HPLC. After completion of the reaction, the mixture was cooled and washed with DM Water (3×1.5 L) at 50-70° C. The organic layer was concentrated to an oil to yield 0.19 kg of 4-[2-(4-fluorophenyl)-5,5-dimethyl-[1,3]-dioxan-2-yl]-butyric acid-3-hydroxy-2,2-dimethylpropyl ester.

### Example-6k

To a 4-necked round-bottomed flask fitted with a thermometer pocket were added 4-(4-fluorophenyl)-4-oxo-butyric acid (0.1 kg, 0.47 mol), cyclo hexane (0.5 L), neopentyl glycol (0.496 kg, 4.72 mol) and pyridinium hydrobromide (0.0076 kg, 0.047 mol) at 20-25° C. The resulting mixture was then refluxed for 3 hours at 80-85° C. The reaction was monitored by TLC/HPLC. After completion of the reaction, the mixture was cooled and washed with DM Water (3×1.5 L) at 50-70° C. The organic layer was concentrated to an oil to yield 0.18 kg of 4-[2-(4-fluorophenyl)-5,5-dimethyl-[1,3]-dioxan-2-yl]-butyric acid-3-hydroxy-2,2-dimethylpropyl ester.

### Example 61

To a 4-necked round-bottomed flask fitted with a thermometer pocket were added 4-(4-fluorophenyl)-4-oxo-butyric acid (0.1 kg, 0.47 mol), cyclohexane (0.5 L), neopentyl glycol (0.496 kg, 4.72 mol) and pyridinium p-toluene sulfonate (0.012 kg, 0.047 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 20-25° C. The organic layer was concentrated to an oil to yield 0.17 kg of 4-[2-(4-fluorophenyl)-5,5-dimethyl-[1,3]-dioxan-2-yl]-butyric acid-3-hydroxy-2,2-dimethylpropyl ester.

### Example-6m

To a 4-necked round-bottomed flask fitted with a thermometer pocket were added methyl-4-(4-fluorophenyl)-4-oxo-butyrate (0.1 kg, 0.44 mol), cyclohexane (0.5 L), neopentyl glycol (0.465 kg, 4.46 mol) and p-toluene sulfonic acid (0.009 kg, 0.047 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 50-70° C. The organic layer was then concentrated to an oil to yield 0.13 kg of 4-[2-(4-fluorophenyl)-5,5-dimethyl-[1,3]-dioxan-2-yl]-butyric acid methyl ester.

### Example-6n

To a 4-necked round-bottomed flask fitted with a thermometer pocket were added methyl-4-(4-fluorophenyl)-4-oxo-butyrate (0.1 kg, 0.44 mol), cyclohexane (0.5 L), neopentyl glycol (0.465 kg, 4.46 mol) and pyridinium hydrobromide (0.0071 kg, 0.044 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 80-85° C. The organic layer was then concentrated to an oil to yield 0.12 kg of 4-[2-(4-fluorophenyl)-5,5-dimethyl-[1,3]-dioxan-2-yl]-butyric acid methyl ester.

### Example-6o

To a 4-necked, round-bottomed flask fitted with a thermometer pocket were added methyl-4-(4-fluorophenyl)-4-oxo-butyrate (0.1 kg, 0.44 mol), cyclohexane (0.5 L), Neopentyl glycol (0.465 kg, 4.46 mol) and pyridinium p-toluene sulfonate (0.011 kg, 0.043 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 50-70° C. The organic layer was then concentrated to an oil to yield 0.12 kg of 4-[2-(4-fluorophenyl)-5,5-dimethyl-[1,3]-dioxan-2-yl]-butyric acid methyl ester.

### Example-6p

To a 4-necked round-bottomed flask fitted with a thermometer pocket were added ethyl-4-(4-fluorophenyl)-4-oxo-butyrate (0.1 kg, 0.42 mol), cyclohexane (0.5 L), neopentyl glycol (0.437 kg, 4.20 mol) and p-toluene sulfonic acid (0.008 kg, 0.042 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 50-70° C. The organic layer was then concentrated to an oil to yield 0.12 kg of 4-[2-(4-fluorophenyl)-5,5-dimethyl-[1,3]-dioxan-2-yl]-butyric acid ethyl ester.

### Example-6q

To a 4-necked round-bottomed flask fitted with a thermometer pocket were added ethyl-4-(4-fluorophenyl)-4-oxo-butyrate (0.1 kg, 0.42 mol), cyclohexane (0.5 L), neopentyl glycol (0.465 kg, 4.46 mol) and pyridinium hydrobromide (0.0067 kg, 0.042 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 50-70° C. The organic layer was then concentrated to an oil to yield 0.11 kg of 4-[2-(4-fluorophenyl)-5,5-dimethyl-[1,3]-dioxan-2-yl]-butyric acid ethyl ester.

### Example-6r

To a 4-necked round-bottomed flask fitted with a thermometer pocket were added ethyl-4-(4-fluorophenyl)-4-oxo-butyrate (0.1 kg, 0.44 mol), cyclohexane (0.5 L), neopentyl glycol (0.465 kg, 4.46 mol) and pyridinium p-toluene sulfonate (0.011 kg, 0.042 mol) at 20-25° C. The resulting mixture was then refluxed for 3 hours at 80-85° C. The reaction was monitored by TLC/HPLC. After completion of the reaction, the mixture was cooled and washed with DM water (3×1.5 L) at 50-70° C. The organic layer was then concentrated to an oil to yield 0.10 kg of 4-[2-(4-fluorophenyl)-5,5-dimethyl-[1,3]-dioxan-2-yl]-butyric acid ethyl ester.

### Synthesis of Intermediate-VII (4-[2-(4-fluorophenyl)-5,5-dimethyl-[1,3]-dioxan-2-yl]-butyric acid)

### Example-7a

To a 4-necked round-bottomed flask fitted with a thermometer pocket were added 4-[2-(4-fluorophenyl)-5,5-dimethyl-[1,3]-dioxan-2-yl]-butyric acid-3-hydroxy-2,2-dimethylpropyl ester (0.1 kg, 0.26 mol), THF (3.5 L), water (2.5 L), and lithium hydroxide mono hydrate (0.015 kg, 0.37 mol) at 20-25° C. The resulting reaction mixture was then stirred for 3 hours. The reaction was monitored by TLC/HPLC. After completion of the reaction, the mixture was cooled to 0° C. and 1N HCl (0.35 L) was added. The mixture was then stirred for 30 min at 20-25° C., extracted with ethyl acetate (2×0.5 L) and washed with DM water (2×0.5 L). the organic layer was then concentrated to afford 0.058 kg of 4-[2-(4-fluorophenyl)-5,5-dimethyl-[1,3]-dioxan-2-yl]-butyric acid.

### Example-7b

To a 4-necked round-bottomed flask fitted with a thermometer pocket were added 4-[2-(4-fluorophenyl)-5,5-dimethyl-[1,3]-dioxan-2-yl]-butyric acid-3-hydroxy-2,2-dimethylpropyl ester (0.1 kg, 0.26 mol), methanol (3.5 L), water (2.5 L), and Sodium hydroxide (0.01 kg, 0.26 mol) at 20-25° C. The resulting mixture was then and stirred for 3 hours. The reaction was monitored by TLC/HPLC. After completion of the reaction, the mixture was cooled to 0° C. and 1N HCl (0.35 L) was added. The mixture was then stirred for 30 minutes at 20-25° C., extracted with ethyl acetate (2×0.5 L), and washed with DM water (2×0.5 L). The organic layer was then concentrated to afford 0.058 Kg of 4-[2-(4-fluorophenyl)-5,5-dimethyl-[1,3]-dioxan-2-yl]-butyric acid.

### Example-7c

To a 4-necked round-bottomed flask fitted with a thermometer pocket were added 4-[2-(4-fluorophenyl)-5,5-dimethyl-[1,3]-dioxan-2-yl]-butyric acid-3-hydroxy-2,2-dimethylpropyl ester (0.1 kg, 0.26 mol), methanol (3.5 L), water (2.5 L), and potassium hydroxide (0.015 kg, 0.26 mol) at 20-25° C. The resulting mixture was then stirred for 3 hours. The reaction was monitored by TLC/HPLC. After completion of the reaction, the mixture was cooled to 0° C. and 1N HCl (0.35 L) was added. The mixture was then stirred for 30 min at 20-25° C., extracted with ethyl acetate (2×0.5 L), and washed with DM water (2×0.5 L). The organic layer was then concentrated to afford 0.058 kg of 4-[2-(4-fluorophenyl)-5,5-dimethyl-[1,3]-dioxan-2-yl]-butyric acid.

### Synthesis of Intermediate-XV (4(S)-(4-hydroxyphenyl)-1-(4-fluorophenyl)-3(R)-{2-[2-(4-fluorophenyl)-5,5-dimethyl-[1,3]-dioxan-2-yl]-ethyl}azetidine-2-one)

### Example-8

To a solution of 4(S)-(4-Benzyloxyphenyl)-1-(4-fluorophenyl)-3(R)-{2-[2-(4-fluorophenyl)-5,5-dimethyl-1,3-dioxan-2-yl]-ethyl}azetidine-2-one (0.1 kg, 0.17 mol) in ethanol (1.2 L) was added 10% Pd/C (10 g) at 20-25° C. The resulting mixture was then stirred for 4 hours under H₂ pressure (60 psi) at 30-35° C. The reaction was monitored by TLC/HPLC. After completion of the reaction, the mixture was filtered and the filer cake was washed with ethanol (0.2 L). The filtrate was then concentrated to afford 0.061 kg of 4(S)-(4-hydroxyphenyl)-1-(4-fluorophenyl)-3(R)-{2-[2-(4-fluorophenyl)-5,5-dimethyl-[1,3]-dioxan-2-yl]-ethyl}azetidine-2-one.

### Synthesis of Intermediate-XVI (4(S)-(4-hydroxyphenyl)-1-(4-fluorophenyl)-3(R)-[3-(4-fluorophenyl)-3oxopropyl]azetidine-2-one)

### Example-9a

To a 4-necked, round-bottomed flask fitted with a thermometer pocket were added 4(S)-(4-hydroxyphenyl)-1-(4-fluorophenyl)-3(R)-{2-[2-(4-fluorophenyl)-5,5-dimethyl-[1,3]-dioxan-2-yl]-ethyl}azetidine-2-one (0.1 kg, 0.20 mol) and formic acid (1.5 L). The resulting mixture was then stirred for 1 hour at 20-25° C. The reaction was monitored by TLC/HPLC. After completion of the reaction, the mixture was concentrated under vacuum to afford an oil. The oil was then dissolved in dichloromethane (0.5 L) and washed with DM water (3×0.5 L). The organic layer was concentrated to an oil, which was crystallized from IPA to afford compound of formula XVI. Yield 0.06 kg of 4(S)-(4-hydroxyphenyl)-1-(4-fluorophenyl)-3(R)-[3-(4-fluorophenyl)-3oxopropyl]azetidine-2-one.

### Example-9b

To a 4-necked, round-bottomed flask fitted with a thermometer pocket were added 4(S)-(4-trimethylsilanyloxyl)-1-(4-fluorophenyl)-3(R)-{2-[2-(4-fluorophenyl)-5,5-dimethyl-[1,3]-dioxan-2-yl]-ethyl}azetidine-2-one (0.1 kg, 0.177 mol) and formic acid (1.5 L). The resulting mixture was then stirred for 1 hour at 20 to 25° C. The reaction was monitored by TLC/HPLC. After completion of the reaction, the mixture was concentrated under vacuum to an oil. The oil was then dissolved in dichloromethane (0.5 L) and washed with DM Water (3×0.5 L). The organic layer was concentrated to an oil, which was crystallized from IPA to afford 0.070 kg of 4(S)-(4-hydroxyphenyl)-1-(4-fluorophenyl)-3(R)-[3-(4-fluorophenyl)-3oxopropyl]azetidine-2-one.

### Example-9c

To a 4-necked, round-bottomed flask fitted with a thermometer pocket was added 4(S)-(4-hydroxyphenyl)-1-(4-fluorophenyl)-3(R)-{2-[2-(4-fluorophenyl)-5,5-dimethyl-[1,3]-dioxan-2-yl]-ethyl}azetidine-2-one (0.1 kg, 0.20 mol) in methylene chloride (0.15 L) formic acid (0.2 L). The resulting mixture was then stirred for 4 hour at 25-30° C. The reaction was monitored by TLC/HPLC. After completion of the reaction, the mixture was washed with DM water (3×0.5 L). The organic layer was concentrated to an oil, which was crystallized from ethanol to afford compound of formula XVI. Yield 0.06 kg of 4(S)-(4-hydroxyphenyl)-1-(4-fluorophenyl)-3(R)-[3-(4-fluorophenyl)-3oxopropyl]azetidine-2-one.

### Example 10

### Reduction of 1-(4-fluorophenyl)-3(R)-[3-(4-fluorophenyl)-3-oxopropyl]-4(S)-(4-hydroxyphenyl)-2-azetidinone (“EZT-ketone”) with (S,S)-i-Pr—SO₂DPEN-ruthenium

“DPEN”=diphenylethylenediamine

[RuCl₂(p-cymene)]₂ (0.0025 mmol, 1.5 mg) and (S,S)-i-Pr—SO₂DPEN (0.006 mmol, 1.9 mg) were weighed in the glass tube of a Radlyes carousel, dissolved in 1 mL methyl tert butyl ether (MTBE) and stirred under nitrogen for one hour at 40° C. To this was added a solution of EZT-ketone (0.25 mmol) in 1 mL of MTBE, followed by 0.5 mL of HCOOH/Et₃N 2M:2M (d=1.01, MW=147, ˜14 equivalents). The reaction was stirred for 17 hours at 65° C. A sample was diluted in MeOH and analyzed by HPLC. The sample contained no starting material, i.e., EZT-ketone, and 96% (by area percent HPLC) of ezetimibe (“EZT”) and the (R,R,S) diastereoisomer of ezetimibe (“(R,R,S) diastereoisomer”), with a diastereomeric excess of EZT (d.e.) of 81%. The integration data from HPLC were not corrected for the response factor.

### Examples 11-44

### Reduction of EZT-Ketone with Sulfonyl-Diamine-Ruthenium Catalysts

The same procedure as described in Example 10 was used, but with a different [RuCl₂(arene)]₂ ligand (0.0025 mmol) and sulfonyl-diamine ligand (0.006 mmol), and with different organic solvents. The samples were analyzed by HPLC in the same manner set forth in Example 10 and were not corrected for the response factor. The results are reported in the table below.

| | | | | Ezetimibe + | | |
|---|---|---|---|---|---|---|
| | | | EZT | (R,R,S) | | |
| | | | ketone | Diastereomer | By-prod. | |
| Ex. | Catalyst | Solvent | (%) | (%) | (%) | d.e. (%) |
| 11. | [RuCl₂ (p-cymene)]₂ + | MTBE | — | 96 | 4 | 72 |
| | (S,S)-MsDPEN | | | | | |
| 12. | [RuCl₂ (hexaMe-benzene)]₂ + | MTBE | — | 81 | 19 | 84 |
| | (S,S)-MsDPEN | | | | | |
| 13. | [RuCl₂ (benzene)]₂ + | MTBE | 3 | 94 | 3 | 70 |
| | (S,S)-MsDPEN | | | | | |
| 14. | [RuCl₂ (p-cymene)]₂ + | MTBE | 10 | 86 | 4 | 64 |
| | (S,S)-MesithylSO₂-DPEN | | | | | |
| 15. | [RuCl₂ (p-cymene)]₂ + | MTBE | — | 96 | 4 | 81 |
| | (S,S)-i-PrSO₂-DPEN | | | | | |
| 16. | [RuCl₂ (p-cymene)]₂ + | MTBE | — | 93 | 7 | 64 |
| | (S,S)-2-naphthyl-DPEN | | | | | |
| 17. | [RuCl₂ (p-cymene)]₂ + | MTBE | — | 95 | 5 | 79 |
| | (S,S)-n-Butyl-DPEN | | | | | |
| 18. | [RuCl₂ (mesitylene)]₂ + | MTBE | — | 74 | 26 | 78 |
| | (S,S)-Ms-DPEN | | | | | |
| 19. | [RuCl₂ (p-cymene)]₂ + | AcOEt | — | 91 | 9 | 73 |
| | (S,S)-Ms-DPEN | | | | | |
| 20. | [RuCl₂ (p-cymene)]₂ + | Toluene | — | 67 | 33 | 81 |
| | (S,S)-Ms-DPEN | | | | (25) | |
| 21. | [RuCl₂ (p-cymene)]₂ + | IPA | — | — | >95 | — |
| | (S,S)-Ms-DPEN | | | | | |
| 22. | [RuCl₂ (mesitylene)]₂ + | MTBE | — | 91 | 9 | 89 |
| | (S,S)-iPr—SO₂-DPEN | | | | | |
| 23. | [RuCl₂ (mesitylene)]₂ + | AcOEt | — | 83 | 17 | 88 |
| | (S,S)-iPr—SO₂-DPEN | | | | | |
| 24. | [RuCl₂ (p-cymene)]₂ + | AcOEt | — | 81 | 19 | 81 |
| | (S,S)-iPr—SO₂-DPEN | | | | | |
| 25. | [RuCl₂ (hexaMe-benzene)]₂ + | AcOEt | 27 | 53 | 20 | 81 |
| | (S,S)-iPr—SO₂-DPEN | | | | | |
| 26. | [RuCl₂ (p-cymene)]₂ + | AcOEt | — | 84 | 16 | 79 |
| | (S,S)-n-Bu-SO₂-DPEN | | | | | |
| 27. | [RuCl₂ (mesitylene)]₂ + | MTBE | — | 91 | 9 | 85 |
| | (S,S)-n-Bu-SO₂-DPEN | | | | | |
| 28. | [RuCl₂ (mesitylene)]₂ + | AcOEt | — | 83 | 17 | 84 |
| | (S,S)-n-Bu-SO₂-DPEN | | | | | |
| 29. | [RuCl₂ (hexaMe-benzene)]₂ + | MTBE | — | 91 | 9 | 91 |
| | (S,S)-n-Bu-SO₂-DPEN | | | | | |
| 30. | [RuCl₂ (p-cymene)]₂ + | MTBE | — | 90 | 10 | 70 |
| | (S,S)-Bn-SO₂-DPEN | | | | | |
| 31. | [RuCl₂ (p-cymene)]₂ + | MTBE | — | 95 | 5 | 88 |
| | (S,S)—Cs-DPEN | | | | | |
| 32. | [RuCl₂ (p-cymene)]₂ + | MTBE | — | 75 | 25 | 62 |
| | (S,S)-3,4-diMe-Ph-SO₂- | | | | | |
| | DPEN | | | | | |
| 33. | [RuCl₂ (p-cymene)]₂ + | MTBE | 1 | 82 | 17 | 67 |
| | (S,S)-1-Naphthyl-DPEN | | | | | |
| 34. | [RuCl₂ (p-cymene)]₂ + | MTBE | 16 | 70 | 14 | 47 |
| | (S,S)-1,3,5-tri-iPr-Ph-DPEN | | | | | |
| 35. | [RuCl₂ (p-cymene)]₂ + | MTBE | — | 90 | 10 | 65 |
| | (S,S)-3,4-diMeO-PhSO₂- | | | | | |
| | DPEN | | | | | |
| 36. | [RuCl₂ (mesitylene)]₂ + | MTBE | 5 | 85 | 10 | 39 |
| | (S,S)—CF₃—SO₂-DPEN | | | | | |
| 37. | [RuCl₂ (p-cymene)]₂ + | MTBE | 1 | 95 | 4 | 67 |
| | (S,S)-pClPh-SO₂-DPEN | | | | | |
| 38. | [RuCl₂ (p-cymene)]₂ + | MTBE | — | 96 | 4 | 65 |
| | (S,S)-pPh-Ph-SO₂-DPEN | | | | | |
| 39. | [RuCl₂ (p-cymene)]₂ + | MTBE | — | 90 | 10 | 60 |
| | (S,S)-pCF₃-Ph-SO₂-DPEN | | | | | |
| 40. | [RuCl₂ (p-cymene)]₂ + | MTBE | — | 92 | 8 | 60 |
| | (S,S)-pNO₂-Ph-SO₂-DPEN | | | | | |
| 41. | [RuCl₂ (p-cymene)]₂ + | MTBE | — | 91 | 9 | 65 |
| | (S,S)-pMeO-Ph-SO₂-DPEN | | | | | |
| 42. | [RuCl₂ (p-cymene)]₂ + | MTBE | 2 | 58 | 40 | 7 |
| | (S,S)-Ms-DACH | | | | | |
| 43. | [RuCl₂ (p-cymene)]₂ + | MTBE | — | 86 | 14 | 60 |
| | (S,S)-1-Naphtyl-SO₂-DACH | | | | | |
| 44. | (S,S)-Tethered-RuCl | MTBE | 50 | 46 | 4 | 81 |

### Example 46

### Reduction of EZT-ketone with (S,S)-Ms-DPEN-rhodium catalyst

[RhCl₂Cp*]₂ (0.0025 mmol, 1.5 mg) and (S,S)-Ms-DPEN (0.006 mmol, 1.7 mg) were weighed in the glass tube of a Radlyes carousel, dissolved in 1 mL MTBE and stirred under nitrogen for one hour at 30° C. To this was added a solution of EZT-ketone (0.25 mmol) in 1 mL of MTBE, followed by 0.5 mL of HCOOH/Et₃N 2M:2M (d=1.01, MW=147, ˜14 equivalents). The reaction was stirred for 18 hours at 30° C. After one hour a sample was taken, diluted in MeOH and analyzed by HPLC: the sample contained 40% starting material and 56% EZT+(R,R,S) diastereoisomer with a d.e. of 91%. After 18 hours HPLC analysis showed no starting material and 95% EZT+(R,R,S) diastereoisomer with a d.e. of 90%. The integration data from HPLC were not corrected for the response factor.

### Example 47

### Reduction of EZT-ketone with (S,S,S)—Cs-DPEN-rhodium catalyst

[RhCl₂Cp*]₂ (0.0025 mmol, 1.5 mg) and (S,S,S)—Cs-DPEN (0.006 mmol, 2.6 mg) were weighed in the glass tube of a Radlyes carousel, dissolved in 1 mL MTBE and stirred under nitrogen for one hour at 30° C. To this was added a solution of EZT-ketone (0.25 mmol) in 1 mL of MTBE, followed by 0.5 mL of HCOOH/Et₃N 2M:2M (d=1.01, MW=147, ˜14 equivalents). The reaction was stirred for 18 hours at 30° C. After one hour a sample was taken, diluted in MeOH and analyzed by HPLC: the sample contained 17% starting material and 79% EZT+(R,R,S) diastereoisomer with a d.e. of 96%. After 18 hours HPLC analysis showed no starting material and 97% EZT+(R,R,S) diastereoisomer with a d.e. of 96%. The integration data from HPLC were not corrected for the response factor.
Transfer Hydrogenation of EZT Ketone with Ruthenium Catalysts

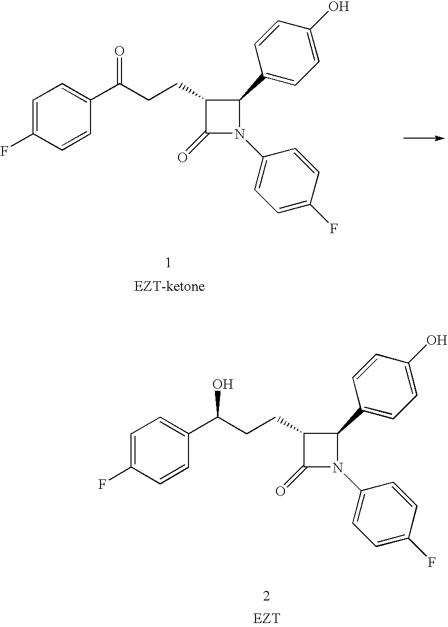

### Example 48

### Transfer hydrogenation of EZT-ketone with (S,S)-i-Bu-SO₂-DPEN-ruthenium catalyst

[RuCl₂(mesitylene)]₂ (1.1 mg, 0.002 mmol) and (S,S)-i-Bu-SO₂-DPEN (1.5 mg, 0.0044 mmol) were weighed in a Schlenk tube with a magnetic stirring bar and a deep gas inlet (below the solvent surface) for sparging with nitrogen and acting as baffle. The solids were placed under nitrogen and 5 mL of AcOEt were added. The solution was stirred at room temperature for 1 hour, then solid EZT-ketone (950 mg, ˜2 mmol considering that the substrate contains 1 equivalent of 2-propanol) was added, followed by 4 mL of a sodium formate solution (5M, pH 6.4 obtained by addition of HCOOH) and 1 mL of water. The reaction was heated to 65° C. and stirred at the fastest rate to achieve the best mixing of the two phases. Sampling after 4 hours showed, by HPLC analysis, 90% EZT+(R,R,S) diastereoisomer (85% d.e.), 7% starting material and 3% by-products. Sampling after 6 hours showed, by HPLC analysis, 92% EZT+(R,R,S) diastereoisomer (85% d.e.), 0.5% starting material and 7.5% by-products. The reaction was stopped and diluted with 5 mL of AcOEt. The organic layer was separated and the aqueous layer was washed with AcOEt (2×5 mL). The combined organic solutions were dried over sodium sulfate and evaporated to give an off-white solid (772 mg, assuming some 0.25 eq. AcOEt: 93% mass recovery). ¹H NNR showed no starting material and ˜10% by-products.

500 mg of the isolated reaction mixture from above was dissolved in MeOH (2 mL) and then a few drops of water were added until a white solid precipitated. The suspension was heated to reflux to give a clear yellow solution that was allowed to cool to room temperature under stirring. The resulting white solid was collected and analyzed by HPLC: 96.5% EZT+(R,R,S) diastereoisomer with 89% d.e. The solid was dissolved in 2 mL of MeOH (at reflux) and then cooled in an ice bath. The solid precipitate was collected, dried under vacuum (150 mg, 50% mass recovery) and analyzed by HPLC: 94% EZT+(R,R,S) diastereoisomer with 92% d.e.

### Example 49

### Transfer hydrogenation of EZT-ketone with (S,S)-i-Pr—SO₂-DPEN-ruthenium catalyst

The same procedure as Example 48 was repeated in reaction using the catalyst derived from [RuCl₂(mesitylene)]₂ (1.1 mg, 0.002 mmol) and (S,S)-i-Bu-SO₂-DPEN (1.4 mg, 0.0044 mmol). After 6 hours reaction time HPLC analysis showed that 3% starting material, 6% by-products and 91% EZT+(R,R,S) diastereoisomer were present. After phase separation a white solid was recovered (assuming 0.25 eq. of AcOEt are present, quantitative mass recovery). ¹H NNR showed no starting material and ˜11% by-products.

### Example 50

### Asymmetric transfer hydrogenation of EZT-ketone with (S,S,S)—Cs-DPEN-rhodium catalyst

(S,S,S)—Cs-DPEN (4.7 mg, 0.011 mmol) and [RhCp*Cl₂]₂ (3.1 mg, 0.005 mmol) were weighed in a Schlenk tube with a magnetic stirring bar and a deep gas inlet (below the solvent surface) for sparging with nitrogen and acting as baffle. The solids were placed under nitrogen and 10 mL of AcOEt and 2 mL of water were added. The solution was stirred at room temperature for 1 hour, then solid EZT-ketone (470 mg, ˜1 mmol considering that the substrate contains one equivalent of 2-propanol) was added, followed by 10 mL of a sodium formate solution (3M, pH 7.8 obtained by addition of HCOOH). The reaction was stirred at room temperature at the fastest rate while nitrogen was bubbled into the mixture. Sampling after 0.5 hours showed, by HPLC analysis, 53% EZT+(R,R,S) diastereoisomer (96% d.e.). Sampling after 1.5 hours showed 86% EZT+(R,R,S) diastereoisomer (96.5% d.e.) and sampling after 4 hours showed >98% EZT+(R,R,S) diastereoisomer (96.5% d.e.). The reaction was stopped and the organic layer was separated. The aqueous layer was washed with more AcOEt (2×10 mL). The combined organic solutions were dried over sodium sulphate and evaporated to give an off-white solid (415 mg, containing 0.25 eq. of AcOEt by ¹H NMR, 97% mass recovery). The solid was dissolved in 2 mL of 2-propanol and water (˜10 mL). The resulting suspension was stirred overnight at room temperature, then the sold was collected on a filter and washed with more water (˜20 mL). After drying under vacuum 287 mg EZT+(R,R,S) diastereoisomer were obtained (67% isolated yield, 97% d.e.).

### Example 51

### Asymmetric Hydrogenation of EZT-BZT-Ketone

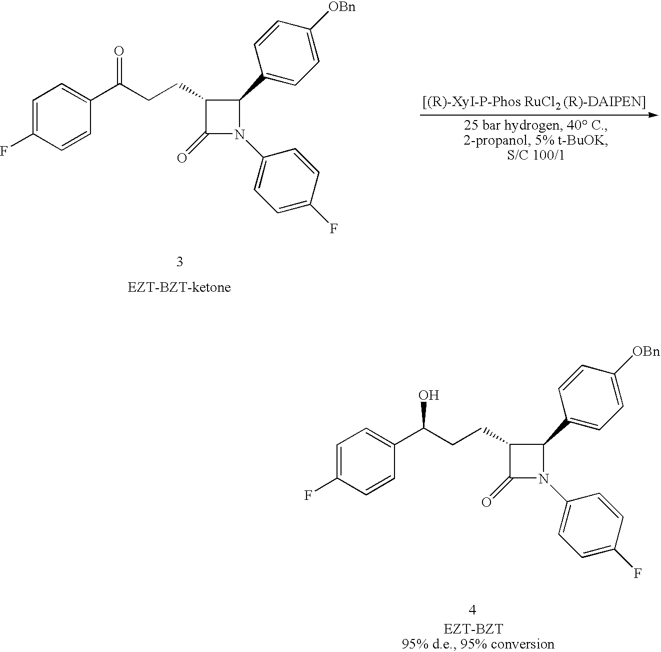

“DAIPEN”=1,1-di(4-anisyl)-2-isopropylethylenediamine

50 mg (1 mmol) of EZT-BZT ketone was weighted in glass vial and placed in an Argonaut Endeavour multi-well autoclave. The vial was purged several times with nitrogen. 1 mL of catalyst solution was prepared by mixing [(R)-XylPPhosRuCl₂(R)-DAIPEN] (12.4 mg, 0.01 mmol), 50 μL t-BuOK (1M in t-BuOH) and 0.95 mL i-PrOH and stirring for 30 min at 60° C. The catalyst solution (Substrate/Base/Catalyst=100/5/1) was added to the substrate, followed by 2 mL of isopropanol. The reaction was purged 5 times with nitrogen and 10 times with hydrogen. The reaction was then stirred at 40° C. under 25 bar of hydrogen for 4 hours. The crude reaction mixture was analyzed by HPLC for conversion and for diastereoselectivity: 95% EZT+(R,R,S) diastereoisomer and d.e. 95%.

What is claimed is:

1. A compound of Formula XII: 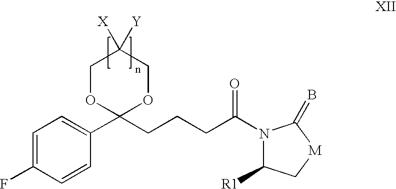
wherein X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl; and
R₂ is hydrogen or a substituted or unsubstituted C₁₋₈ alkyl, with the proviso that X and Y are not both hydrogen.

2. A process for preparing a compound of Formula XII: 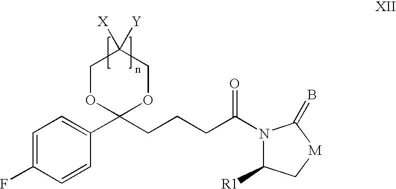
comprising combining the compound of Formula XIII: 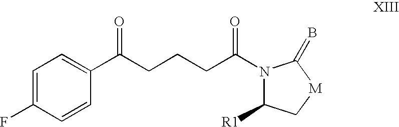
with a diol derivative of Formula III: 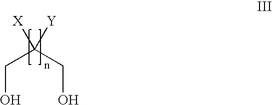
to obtain the compound of Formula XII,
wherein X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl; and
R₂ is hydrogen or a substituted or unsubstituted C₁₋₈ alkyl.

3. (canceled)

4. (canceled)

5. A process for preparing a compound of Formula XII: 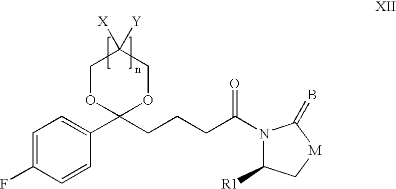
comprising combining a compound of Formula VIII: 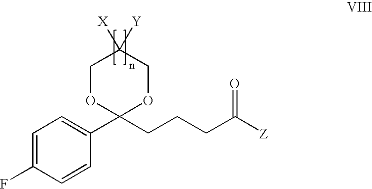
with a chiral auxiliary of Formula IX: 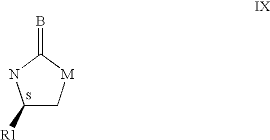
to form the compound of claim 1, wherein
X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl;
R₂ is hydrogen or a substituted or unsubstituted C₁₋₈ alkyl;
Z is a halogen, an activated ester of a carboxylic acid, —O—CO—R or —O—COOR; and
R is C₁₋₈ alkyl.

6. A process for preparing a compound of Formula XII: 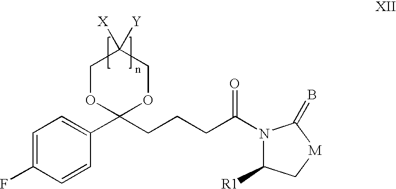
comprising:
(a) reacting a 4-fluorobenzoyl butyric acid or ester of Formula V 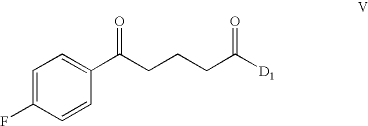
with a diol derivative of Formula III: 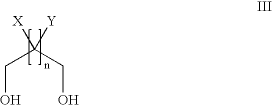
to form a compound of Formula VI: 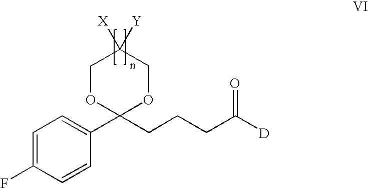
(b) reacting the compound of Formula VI with an inorganic base to obtain a compound of Formula VII: 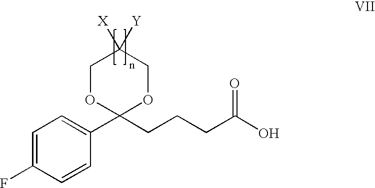
(c) converting the compound of Formula VII to a compound of Formula VIII: 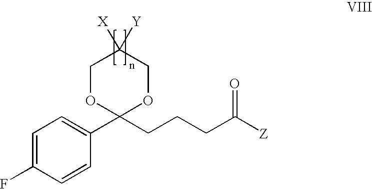
and
(d) reacting the compound of Formula VIII with a chiral auxiliary of Formula IX: 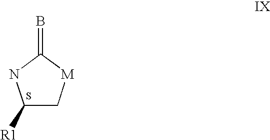
to obtain a compound of Formula XII, wherein
X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl;
R₂ is hydrogen or a substituted or unsubstituted C₁₋₈ alkyl;
Z is a halogen, an activated ester of a carboxylic acid, —O—CO—R or —O—COOR; and
R is C₁₋₈ alkyl;
D is substituted or unsubstituted C₁₋₈ alkoxy; and
D₁ is hydroxyl, or substituted or unsubstituted C₁₋₈ alkoxy.

7. (canceled)

8. A compound of Formula VI: 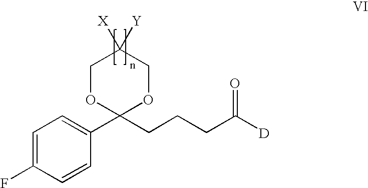
wherein X is hydrogen or a substituted or unsubstituted C₁₋₈ alkyl;
Y is hydrogen or a substituted or unsubstituted C₁₋₈ alkyl;
n is an integer between 0 and 3; and
D is substituted or unsubstituted C₁₋₈ alkoxy.

9. (canceled)

10. (canceled)

11. A compound of Formula VII: 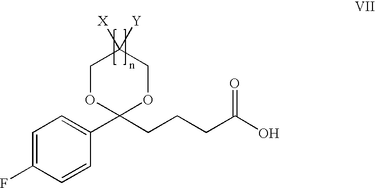
wherein X is hydrogen or a substituted or unsubstituted C₁₋₈ alkyl;
Y is hydrogen or a substituted or unsubstituted C₁₋₈ alkyl; and
n is an integer between 0 and 3.

12. (canceled)

13. A compound of Formula VIII: 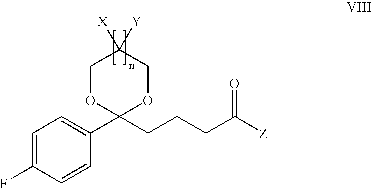
wherein X is hydrogen or a substituted or unsubstituted C₁₋₈ alkyl;
Y is hydrogen or a substituted or unsubstituted C₁₋₈ alkyl;
n is an integer between 0 and 3;
Z is a halogen, an activated ester of a carboxylic acid, —O—CO—R or —O—COOR; and
R is C₁₋₈ alkyl.

14. (canceled)

15. A compound of Formula X: 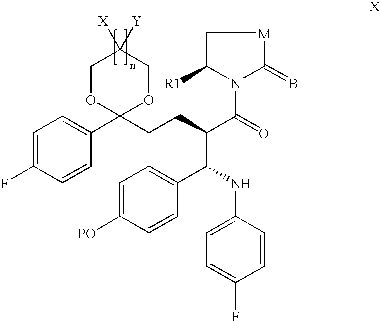
wherein X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl;
R₂ is hydrogen or a substituted or unsubstituted C₁₋₈ alkyl; and
P is a hydroxyl protecting group, with the proviso that X and Y are not both hydrogen.

16. (canceled)

17. (canceled)

18. A process for preparing a compound of Formula X: 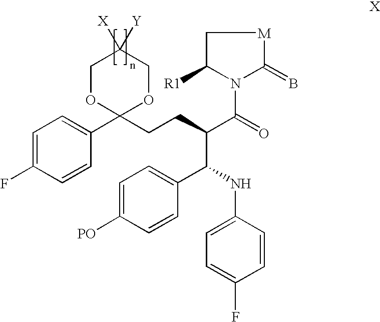
comprising
(a) reacting a compound of Formula XII: 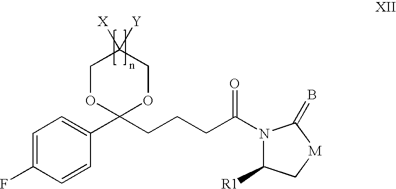
with a compound of Formula XI: 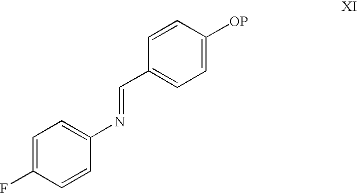
in the presence of a tertiary organic base and at least one acid catalyst selected from the group consisting of para-toluene sulfonic acid, methane sulfonic acid, benzene sulfonic acid, C₁₋₄ tri-alkylsilyl chloride, titanium tetrachloride, titanium tetra isopropoxide, aluminum chloride, zinc chloride, and BF₃-etherate to obtain the compound of Formula X, wherein
X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl;
R₂ is hydrogen or a substituted or unsubstituted C₁₋₈ alkyl; and
P is a hydroxyl protecting group.

19-21. (canceled)

22. A compound of Formula IV: 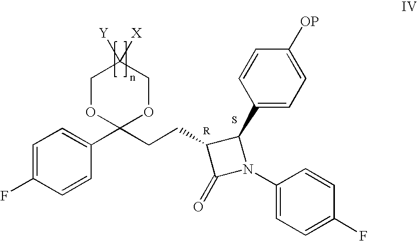
wherein X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group, with the proviso that X and Y are not both hydrogen.

23. The compound of claim 22, wherein P is benzyl, para-methoxybenzyl, or trimethyl silyl.

24. The compound of claim 22, wherein P is benzyl.

25. A process for preparing a compound of Formula IV: 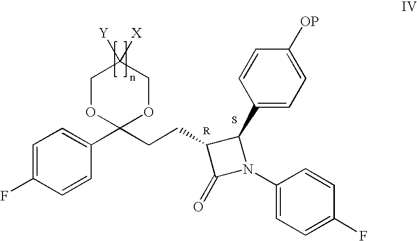
comprising cyclizing a compound of Formula X: 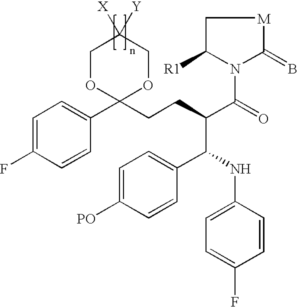
with a silylating agent selected from the group consisting of bis(trimethylsilyl)acetamide, N-methyl-O-trimethylsilyl acetamide, iso-propenyloxy trimethylsilane, bis(trimethylsilyl)urea, hexamethyldisilazane, and a mixture of hexamethyldisilazane and trimethyl silyl chloride, optionally in the presence of a fluoride ion catalyst, to form the compound of Formula IV, wherein
X is hydrogen or a substituted or unsubstituted C₁₋₈ alkyl;
Y is hydrogen or a substituted or unsubstituted C₁₋₈ alkyl;
n is an integer between 0 and 3;
P is a hydroxyl protecting group;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl; and
R₂ is hydrogen or a substituted or unsubstituted C₁₋₈ alkyl.

26. The process of claim 25, wherein the silylating agent is bis(trimethylsilyl)acetamide.

27. The process of claim 25, wherein a fluoride ion catalyst is present and is a fluoride ion catalyst of Formula XVII: 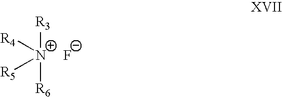
wherein R₃ is a substituted or unsubstituted C₁₋₈ alkyl or C₆₋₁₄ aryl;
R₄ is a substituted or unsubstituted C₁₋₈ alkyl or C₆₋₁₄ aryl;
R₅ is a substituted or unsubstituted C₁₋₈ alkyl or C₆₋₁₄ aryl; and
R₆ is a substituted or unsubstituted C₁₋₈ alkyl or C₆₋₁₄ aryl.

28. A compound of Formula XV: 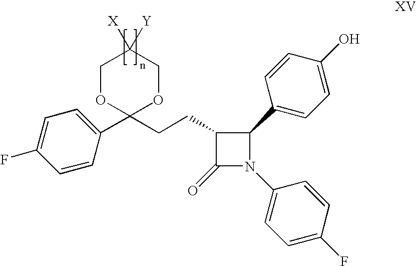
wherein X is hydrogen or a substituted or unsubstituted C₁₋₈ alkyl;
Y is hydrogen or a substituted or unsubstituted C₁₋₈ alkyl; and
n is an integer between 0 and 3, with the proviso that X and Y are not both hydrogen.

29. A process for preparing a compound Formula XV: 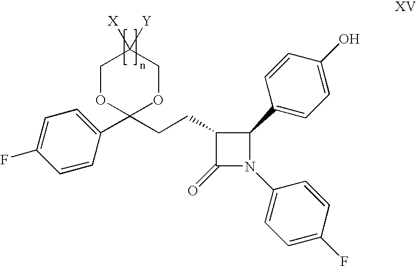
comprising converting a compound of Formula IV: 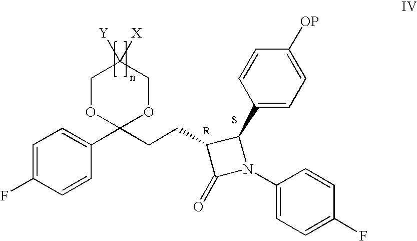
to the compound of Formula XV by removing P using catalytic hydrogenation or hydride transfer reaction,
wherein X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group.

30. A process for preparing a compound Formula XV: 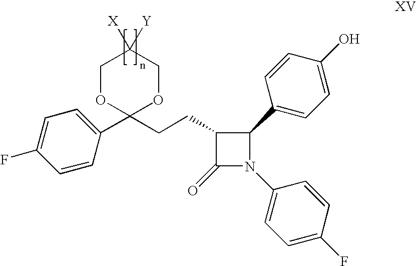
comprising reacting a compound of Formula IV: 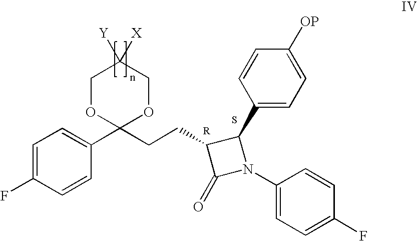
with a quaternary ammonium fluoride in the presence of an organic solvent to obtain the compound of Formula XV,
wherein X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group.

31. A process for preparing a compound of Formula XVI: 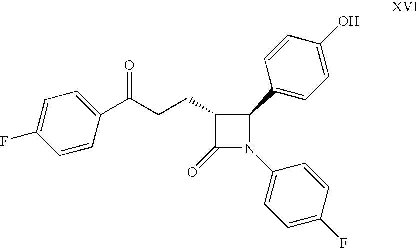
comprising converting a compound of Formula IV: 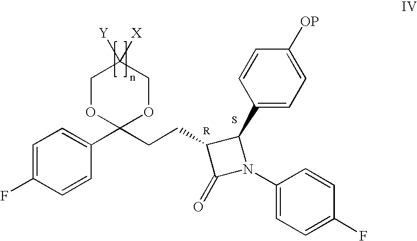
to the compound of Formula XVI using acid hydrolysis, wherein
X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group.

32. A process for preparing a compound of Formula XVI: 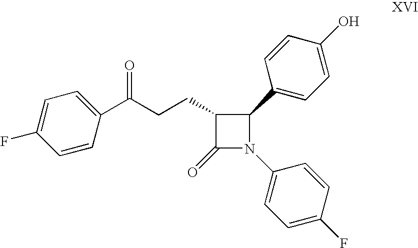
comprising converting the compound of Formula XV: 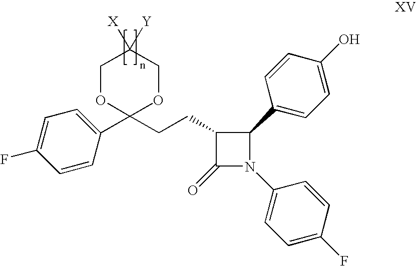
to the compound of Formula XVI in the presence of at least one of an organic acid or a mineral acid, wherein
X is hydrogen or a substituted or unsubstituted C₁₋₈ alkyl;
Y is hydrogen or a substituted or unsubstituted C₁₋₈ alkyl; and
n is an integer between 0 and 3.

33. (canceled)

34. A process for preparing ezetimibe comprising at least one of the following steps:
(a) reacting a compound of Formula XIII: 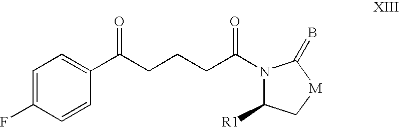
with a diol derivative of Formula III: 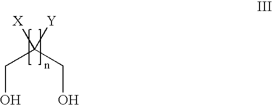
to obtain a compound of Formula XII: 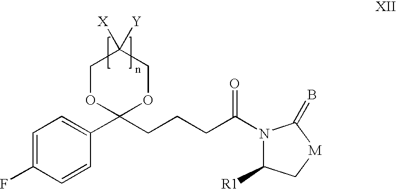
(b) reacting the compound of Formula XII with a compound of Formula XI: 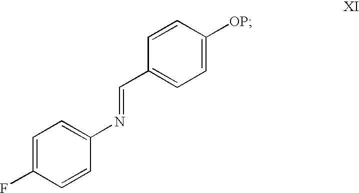
to obtain a compound of Formula X: 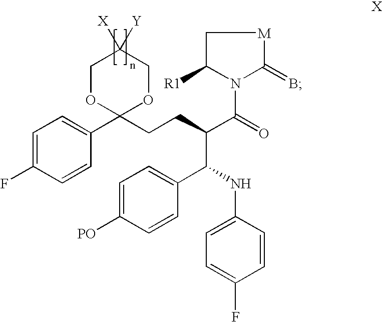
(c) combining the compound of Formula X with a silylating agent and optionally a fluoride ion catalyst of Formula XVII: 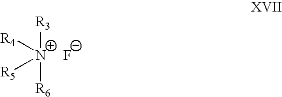
to obtain a compound of Formula IV: 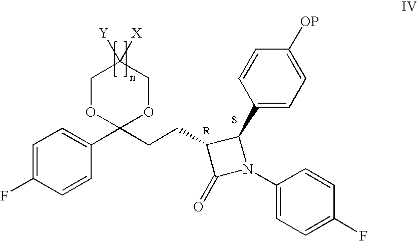
(d) combining the compound of Formula IV with an acid to obtain a compound of Formula II: 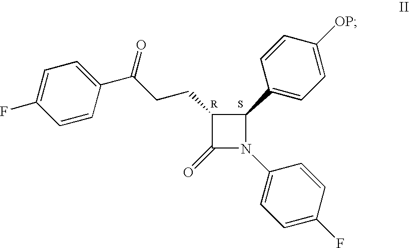
(e) converting the compound of Formula II to the compound of Formula XIV 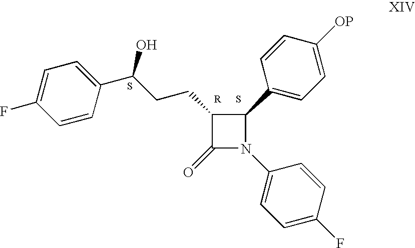
by one of (i) combining the compound of Formula II with at least one chiral reducing agent selected from the group consisting of β-chloro diisopinocamphenyl borane and a borane in the presence of at least one chiral catalyst selected from the group consisting of: (R)-tetrahydro-1-methyl-3,3-diphenyl-1H,3H-pyrrol(1,2-c)(1,3,2)oxaza-borolideine,
(R)-tetrahydro-1-butyl-3,3-diphenyl-1H,3H-pyrrol(1,2-c)(1,3,2)oxaza-borolideine, and
(R)-tetrahydro-1-phenyl-3,3-diphenyl-1H,3H-pyrrol(1,2-c)(1,3,2)oxaza-borolideine,
and optionally at least one solvent selected from the group consisting of:
dichloromethane, tetrahydrofuran, toluene, and tert-butyl methyl ether, to obtain the compound of Formula XIV;
(ii) combining the compound of Formula II with at least one chiral catalyst selected from the group consisting of [[RuCl₂(p-cymene)]₂, (S,S)-i-Pr—SO₂DPEN], [[RuCl₂(p-cymene)]₂(S,S)-MsDPEN], [[RuCl₂(hexaMe-benzene)]₂(S,S)-MsDPEN], [[RuCl₂(benzene)]₂(S,S)-MsDPEN], [[RuCl₂(p-cymene)]₂(S,S)-MesithylSO₂-DPEN], [[RuCl₂(p-cymene)]₂(S,S)-2-naphthyl-DPEN], [[RuCl₂(p-cymene)]₂(S,S)-n-Butyl-DPEN], [[RuCl₂(mesitylene)]₂(S,S)-Ms-DPEN], [[RuCl₂(mesitylene)]₂(S,S)-iPr—SO₂-DPEN], [[RuCl₂(hexaMe-benzene)]₂(S,S)-iPr—SO₂-DPEN], [[RuCl₂(p-cymene)]₂(S,S)-n-Bu-SO₂-DPEN], [[RuCl₂(mesitylene)]₂(S,S)-n-Bu-SO₂-DPEN], [[RuCl₂(hexaMe-benzene)]₂(S,S)-n-Bu-SO₂-DPEN], [[RuCl₂(p-cymene)]₂(S,S)-Bn-SO₂-DPEN], [[RuCl₂(p-cymene)]₂(S,S)—Cs-DPEN], [[RuCl₂(p-cymene)]₂(S,S)-3,4-diMe-Ph-SO₂-DPEN], [[RuCl₂(p-cymene)]₂(S,S)-1-Naphthyl-DPEN], [[RuCl₂(p-cymene)]₂(S,S)-1,3,5-tri-iPr-Ph-DPEN], [[RuCl₂(p-cymene)]₂(S,S)-3,4-diMeO-PhSO₂-DPEN], [[RuCl₂(mesitylene)]₂(S,S)—CF₃—SO₂-DPEN], [[RuCl₂(p-cymene)]₂(S,S)-pClPh-SO₂-DPEN], [[RuCl₂(p-cymene)]₂(S,S)-pPh-Ph-SO₂-DPEN], [[RuCl₂(p-cymene)]₂(S,S)-pCF₃-Ph-SO₂-DPEN], [[RuCl₂(p-cymene)]₂(S,S)-pNO₂-Ph-SO₂-DPEN], [[RuCl₂(p-cymene)]₂(S,S)-pMeO-Ph-SO₂-DPEN], [[RuCl₂(p-cymene)]₂(S,S)-Ms-DACH], [[RuCl₂(p-cymene)]₂(S,S)-1-Naphtyl-SO₂-DACH], [(S,S)-Tethered —RuCl], [[RhCl₂Cp*]₂(S,S)-Ms-DPEN], [[RhCl₂Cp*]₂(S,S,S)—Cs-DPEN], and [[RuCl₂(mesitylene)]₂(S,S)-i-Bu-SO₂-DPEN]; at least one hydrogen source selected from the group consisting of formic acid or a salt thereof, C₃-C₁₃ secondary alcohol, and cyclohexadiene; and an organic solvent to obtain the compound of Formula XIV; or
(iii) combining the compound of Formula II with at a chiral catalyst under an inert gas environment; adding a base to obtain a reaction mixture; and subjecting the reaction mixture to a hydrogen pressure of about 2 bars to about 40 bars to obtain the compound of Formula XIV; or
(f) deprotecting group the compound of Formula XIV to obtain ezetimibe,
wherein X is hydrogen or a substituted or unsubstituted C₁₋₈ alkyl;
Y is hydrogen or a substituted or unsubstituted C₁₋₈ alkyl;
n is an integer between 0 and 3;
B is O or S;
M is O, S, or NR₂;
R₁ is a substituted or unsubstituted C₁₋₈ alkyl, C₆₋₁₄ aryl, C₇₋₁₅ arylalkyl, or C₂₋₉ alkoxycarbonyl;
R₂ is hydrogen or a substituted or unsubstituted C₁₋₈ alkyl;
P is a hydroxyl protecting group;
R₃ is a substituted or unsubstituted C₁₋₈ alkyl or C₆₋₁₄ aryl;
R₄ is a substituted or unsubstituted C₁₋₈ alkyl or C₆₋₁₄ aryl;
R₅ is a substituted or unsubstituted C₁₋₈ alkyl or C₆₋₁₄ aryl; and
R₆ is a substituted or unsubstituted C₁₋₈ alkyl or C₆₋₁₄ aryl.

35. The process of claim 34, wherein the compound of Formula VIII and the diol derivative of Formula III are combined in the presence of at least one of para-toluene sulfonic acid, methane sulfonic acid, benzene sulfonic acid, trimethyl silyl chloride, titanium tetrachloride, titanium tetra isopropoxide, aluminum chloride, zinc chloride, BF₃-etherate, pyridinium hydrobromide, pyridinium paratoluenesulfonate, pyridinium methane sulfonate, pyridinium benzene sulfonate, and triethyl amine hydrochloride.

36. The process of claim 34, wherein the compound of Formula IV is combined with at least one acid selected from the group consisting of: formic acid, acetic acid, propionic acid, camphor sulfonic acid, sulfonic acid, hydrochloric acid, and sulfuric acid to obtain the compound of Formula II.

37. A process for preparing ezetimibe comprising the steps of:
(a) deprotecting a compound of Formula IV: 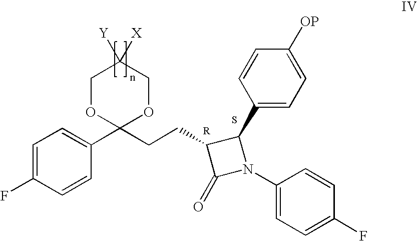
using catalytic hydrogenation, hydride transfer reduction, acid hydrolysis, or reacting with a quaternary ammonium fluoride to obtain a compound of Formula XV: 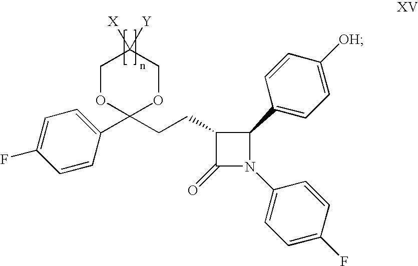
(b) deprotecting the compound of Formula XV with an acid to obtain a compound of Formula XVI: 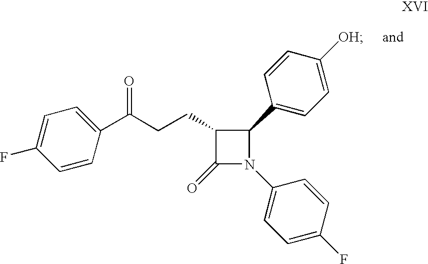
(c) reducing the compound of Formula XVI in the presence of a chiral catalyst to ezetimibe,
wherein X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group.

38. A process for preparing a compound of Formula II: 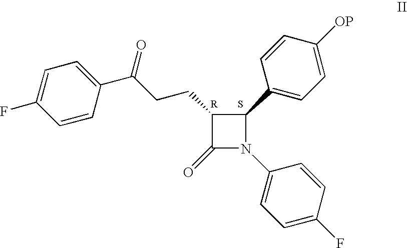
comprising combining a compound of Formula IV: 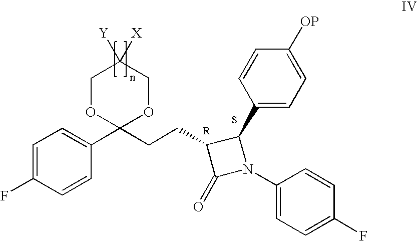
with an acid to obtain the compound of Formula II,
wherein X is hydrogen or a substituted or unsubstituted C₁₋₈ alkyl;
Y is hydrogen or a substituted or unsubstituted C₁₋₈ alkyl;
n is an integer between 0 and 3; and
P is a hydroxyl protecting group.

39. The process of claim 38, wherein the acid is at least one of formic acid, acetic acid, propionic acid, camphor sulfonic acid, hydrochloric acid, or sulfuric acid.

40. Crystalline 3-{4-[2-(4-Fluorophenyl)-5,5-dimethyl-[1,3]dioxan-2-yl]butyryl}-(4S)-phenyl-1,3-oxazolidin-2-one characterized by a PXRD pattern having peaks at about 16.3, 19.5, 20.3, 24.4, and 25.0±0.2 degrees two-theta.

41. (canceled)

42. A compound prepared according to the process of claim 25.

43. A process for preparing an azetidinone comprising converting the compound of claim 42 to the azetidinone.

44. A process for preparing an azetidinone comprising converting the compound of claim 22.

45. The process of claim 43, wherein the azetidinone is ezetimibe.

46. Ezetimibe prepared according to claim 34.

47. A pharmaceutical composition comprising the ezetimibe of claim 46.

48. A method for reducing cholesterol in a mammal comprising administering a therapeutically effective amount of the ezetimibe of claim 46.

49. A method for reducing cholesterol in a mammal comprising administering a therapeutically effective amount of the pharmaceutical composition of claim 47.

50. Use of ezetimibe according to claim 46 for the manufacture of a medicament for reducing cholesterol in a mammal.

51. Use of a compound of claim 22 for the manufacture of ezetimibe.

52. Use of a process of claim 25 for the manufacture of ezetimibe.

53. Use of a diol derivative of Formula III: 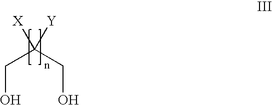
as a carbonyl protecting group for the manufacture of ezetimibe,
wherein X is hydrogen or a substituted or unsubstituted C₁₋₈ alkyl;
Y is hydrogen or a substituted or unsubstituted C₁₋₈ alkyl; and
n is an integer between 0 and 3.
